(19) United States
(12) Patent Application Publication
(10) Pub. No.: US 2019/0344485 A1
(43) Pub. Date: Nov. 14, 2019

(54) Method and apparatus for the manufacture of cellular solids

(71) Applicant: Aron H. Blaesi, (US)

(72) Inventors: Aron H. Blaesi, Cambridge, MA (US); Nannaji Saka, Cambridge, MA (US)

(73) Assignee: Aron H. Blaesi, Cambridge, MA (US)

(21) Appl. No.: 16/246,456

(22) Filed: Jan. 11, 2019

Related U.S. Application Data
(63) Continuation of application No. PCT/US17/41609, filed on Jul. 11, 2017.

(51) Int. Cl. B29C 44/34; B29C 44/46; B29C 44/38; A61K 9/12

(52) U.S. Cl. CPC B29C 44/3449; B29C 44/462; A61K 9/122; B29C 44/386; B29C 44/3415; B29C 44/348

(57) ABSTRACT

Cellular solids combine the properties of the solid material that forms the cell walls and edges with the properties of the matter that fills the cells. This results in unique properties that for many applications are superior to the properties of the single-phase, solid materials. Presented herein, accordingly, are an apparatus and a method for the manufacture of cellular solids with predictable microstructure. The apparatus includes an internally hollow housing defining an extrusion channel, a cell-fluid feeding unit for injecting cell-fluid into the extrusion channel, a conveying element, and a mold or a surface. The method includes injecting cell-fluid into a plasticized matrix in the extrusion channel to form a plasticized foam and dispensing the plasticized foam through an exit port of the extrusion channel into a mold or on a surface.

### CROSS-REFERENCE TO RELATED APPLICATIONS

This application is a continuation of, and incorporates herein by reference in its entirety, the International Application No. PCT/US2017/041609 filed on Jul. 11, 2017 and titled “Method and Apparatus for the Manufacture of Cellular Solids”.

This application is related to, and incorporates herein by reference in their entirety, the commonly owned U.S. application Ser. No. 14/907,891 filed on Jan. 27, 2016 and titled “Melt-Processed Polymeric Cellular Dosage Form”, and the U.S. application Ser. No. 15/482,776 filed on Apr. 9, 2017 and titled “Fibrous dosage form”. This application is further related to, and incorporates herein by reference in its entirety, the International Application No. PCT/US16/58935 filed on Oct. 26, 2016 and titled “Solid Dosage Form for Immediate Drug Release and Apparatus and Method for Manufacture thereof”.

### FIELD OF THE INVENTION

This invention relates generally to methods and apparatuses for the manufacture of cellular solids, and more particularly to methods and apparatuses for the manufacture of cellular solids with predictable microstructure.

### BACKGROUND OF THE INVENTION

Cellular solids generally comprise an interconnected solid structure enclosing chemically or physically distinct compartments or cells. The properties of cellular solids are a combination of the properties of the individual materials they consist of Thus for many applications the cellular solids are superior to any of its single-phase, minimally-porous constituents.

For example, in the commonly owned U.S. patent application Ser. No. 14/907,891 and the publications in J. Control. Release, 220 (2015) 397-405; Eur. J. Pharm. Biopharm, 103 (2016) 210-218; Int. J. Pharm. 509 (2016) 444-453; and Materials Science and Engineering C (2017), the present inventors (Blaesi and Saka) have introduced cellular dosage forms for drug delivery. It was demonstrated that the drug release rate by the cellular dosage forms can be tailored by altering the cell volume fraction and the connectivity of the void space. Specifically, if the cell volume fraction was large and the cell structure predominantly open, the drug release rate by the cellular dosage forms was an order of magnitude greater than that of the corresponding minimally-porous solid material.

In prior work the cellular dosage forms were prepared as follows. A solid disk (consisting of polyethylene glycol (a polymer) and Acetaminophen) was first placed in a container in a high-pressure oven. The disk was melted and charged with an inert gas at a temperature of 70-130° C. and a pressure of 5-34 MPa. After saturation of the molten disk by the gas, the pressure was reduced to induce nucleation, growth, and coalescence of gas bubbles. Finally, the temperature of the mixture was lowered to solidify the cellular structure.

Though adequate for preparing experimental cellular structures, such a process is not optimal for continuous manufacture for various reasons. First, the diffusivity of gas in the polymer at the process temperatures and pressures is of the order of 10⁻¹⁰ to 10⁻⁹ m²/s limiting the rates at which the several millimeters thick raw material can be charged with gas. Second, high pressure is required to add the required amount of gas to the material. Third, relatively high temperatures are needed to achieve the optimal rates of nucleation, growth, and coalescence of the gas bubbles. High temperatures degrade many kinds of organic materials, such as select foods, drugs, or polymers. Fourth, because nucleation is a random process, predictability of the resulting microstructures is limited. Predictable and precisely controlled cell structures, however, would be desirable to optimize the cellular material for a particular application. cell structures.

The above limitations could be overcome if the gas would be delivered directly into the melt stream, eliminating the gas dissolution, bubble nucleation, and bubble growth steps altogether. In this disclosure, therefore, a micro- or milli-fluidic process is presented where bubbles or droplets of a given volume are injected in a moving liquid stream at fixed time intervals and at discrete locations. The process enables continuous manufacture of cellular solids with deterministic microstructures, for both the flow rate of the liquid stream and the size and spacing of the bubbles or droplets (also referred to herein as “cell” or “cells”) are controllable.

### SUMMARY OF THE INVENTION

Thus, in one aspect of the continuous (or Batch) process disclosed herein, one or more granular solids are first injected into an extrusion channel inside a housing. The injected one or more granular solids are then heated to a temperature greater than the melting temperature of at least one injected granular solid. Thus at least one injected granular solid is fluidized upon heating so that the one or more injected granular solids form a plasticized matrix in the extrusion channel. The plasticized matrix is then conveyed towards an exit port of the extrusion channel by the application of mechanical work. As the plasticized matrix transits towards the exit port, cell-fluid is injected into the plasticized matrix in the extrusion channel to form a plasticized foam. The plasticized foam is then dispensed through an exit port into a mold or on a surface. The temperature of the dispensed plasticized foam may subsequently be reduced to below the melting temperature of the plasticized foam to solidify the plasticized foam and form a cellular solid.

In another aspect, one or more granular solids are first injected into an extrusion channel inside a housing. Then a solvent is injected into the extrusion channel to solvate at least one injected granular solid and effect that the one or more injected granular solids form a plasticized matrix in the extrusion channel. The plasticized matrix is then conveyed towards an exit port of the extrusion channel by the application of mechanical work. As the plasticized matrix transits towards the exit port, cell-fluid is injected into the plasticized matrix in the extrusion channel to form a plasticized foam. The plasticized foam is then dispensed through an exit port into a mold or on a surface. In some embodiments, the concentration of solvent in the dispensed plasticized foam may subsequently be reduced to solidify the cellular structure.

In a further aspect, a plasticized matrix is injected into an extrusion channel inside a housing. The plasticized matrix is then conveyed towards an exit port of the extrusion channel by applying mechanical work on the plasticized matrix along the extrusion channel. As the plasticized matrix transits towards the exit port, cell-fluid is injected into the plasticized matrix to form a plasticized foam in the extrusion channel. The plasticized foam is then dispensed through an exit port into a mold or on a surface. In some embodiments, the dispensed plasticized foam may subsequently be solidified.

The apparatus for the manufacture of cellular solids disclosed herein comprises an internally hollow housing having an internal surface encapsulating and defining an extrusion channel having a first end and a second end and a cross section extending axially along its length from said first end to said second end and terminating into an exit port at the second end.

In one aspect, the housing has at least a first feeding port between the first end and second end for injecting one or more granular solids into the extrusion channel and has at least a second feeding port between the first feeding port and an exit port for injecting cell-fluid into the extrusion channel. The apparatus further comprises a granular solid feeding unit for injecting one or more granular solids through the first feeding port into the extrusion channel. The apparatus further comprises at least one heating element for fluidizing at least one injected granular solid so as to form a plasticized matrix in the extrusion channel. The apparatus further comprises a cell-fluid feeding unit attached to the second feeding port for injecting cell-fluid into the plasticized matrix so as to form a plasticized foam in the extrusion channel. The apparatus further comprises a conveying element for extruding the plasticized foam in the extrusion channel through an exit port, and a mold or a surface for accepting the extruded plasticized foam.

In another aspect, the housing has at least a first feeding port between the first end and the second end for injecting one or more granular solids into the extrusion channel, at least a second feeding port between the first feeding port and the exit port for injecting at least one solvent into the extrusion channel so as to form a plasticized matrix in the extrusion channel by solvating at least one injected granular solid, and at least one third feeding port between the second feeding port and an exit port for injecting cell-fluid into the extrusion channel. The apparatus further comprises a granular solid feeding unit for injecting one or more granular solids through the first feeding port into the extrusion channel. The apparatus further comprises a solvent feeding unit attached to the second feeding port for injecting at least one solvent into the extrusion channel. The apparatus further comprises a cell-fluid feeding unit attached to the third feeding port for injecting cell-fluid into the plasticized matrix to form a plasticized foam in the extrusion channel. The apparatus further comprises a conveying element for extruding the plasticized foam in the extrusion channel through an exit port, and a mold or a surface for accepting the extruded plasticized foam.

In yet another aspect, the housing has at least a first feeding port between the first end and the second end for injecting a plasticized matrix into the extrusion channel and at least a second feeding port between the first feeding port and an exit port for injecting cell-fluid into the extrusion channel. The apparatus further comprises a plasticized matrix feeding unit for injecting the plasticized matrix through the first feeding port into the extrusion channel. The apparatus further comprises a cell-fluid feeding unit attached to the third feeding port for injecting cell-fluid into the plasticized matrix to form a plasticized foam in the extrusion channel. The apparatus further comprises a conveying element for extruding the plasticized foam in the extrusion channel through an exit port, and a mold or a surface for accepting the extruded plasticized foam.

Elements of the method and apparatus disclosed herein include but are not limited to the following.

In some embodiments, the extrusion channel cross section is uniform along its length.

In some embodiments, the extrusion channel cross section tapers down before an exit port to the cross section of said exit port.

In some embodiments, the extrusion channel bifurcates into at least one other end comprising an exit port.

In some embodiments, a fraction of the housing is optically transparent.

In some embodiments, the housing comprises at least one other feeding port for injecting cell-fluid into the extrusion channel.

In some embodiments, the heating element is a wrap around heater.

In some embodiments, the heating element is embedded into the housing.

In some embodiments, the cell-fluid injection is performed using a cell-fluid injection unit having a cell-fluid injection channel with a first end and at least a second end contiguous with and terminating in the extrusion channel.

In some embodiments, the volume of the cell-fluid injection channel divided by the number of its second ends contiguous with and terminating in the extrusion channel is no greater than 200 mm³.

In some embodiments, the cell fluid is injected into a flowing plasticized matrix by the application of pressure pulses in the cell-fluid injection channel so as to control the volume of injected cells and the repetition rate of cell injection.

In some embodiments, the device for the application of pressure pulses in the cell-fluid injection channel comprises at least one cell-fluid pump.

In some embodiments, the device for the application of pressure pulses in the cell-fluid injection channel comprises at least an on/off valve, said on/off valve having at least an on-state and at least an off-state, said on-state permitting cell fluid injection from a pressure reservoir into the extrusion channel, said off-state restricting cell fluid injection from said pressure reservoir into the extrusion channel.

In some embodiments, the pressure pulse duration in one repetition period is no greater than 10 seconds.

In some embodiments, the repetition rate of pressure pulses is greater than 2 per minute.

In some embodiments, the cell-fluid injection is performed using at least one flow resistor.

In some embodiments, the greater of the flow resistance of said flow resistor or the sum of the flow resistances of all the flow resistors connected in series is greater than 1×10⁹ Pa·s/m³.

In some embodiments, the cross section along the length of the cell-fluid injection channel is so that the flow resistance of said cell-fluid injection channel is greater than 1×10⁹ Pa·s/m³.

In some embodiments, the cell-fluid is injected into the plasticized matrix during the operation of at least one conveying element.

In some embodiments, the average velocity of the plasticized foam in the extrusion channel with respect to the housing is greater than 0.1 mm/s.

In some embodiments, the mean free distance between the cells in the plasticized foam is no greater than 100 mm (e.g., between 10 μm and 100 mm).

In some embodiments, the shear viscosity of the plasticized matrix in the extrusion channel is greater than 0.005 Pa·s.

In some embodiments, the cell-fluid is a gas.

In some embodiments, the cell-fluid is a liquid.

In some embodiments, the mechanical work is applied using a screw.

In some embodiments, the plasticized foam solidifies in a mold or on a surface.

In some embodiments, the cellular solid comprises one or more solid constituents, said solid constituents comprising a three-dimensional network of walls and edges, said walls and edges defining one or more cells in said cellular structure; wherein the average cell size is 0.1 μm-10 mm; the average wall or edge thickness is 0.1 μm-10 mm; and the volume fraction of cells with respect to a representative control volume of the cellular solid is 0.05-0.95.

In some embodiments, the cellular solid is a pharmaceutical solid dosage form.

Additional elements and variants of the aspects of the method and apparatus disclosed herein are described throughout this specification. Elements of embodiments described with respect to one aspect of the invention can be applied with respect to another aspect. By way of example but not by way of limitation, certain embodiments of the method claims can include features of the apparatus claims, and vice versa.

This invention may be better understood by reference to the accompanying drawings, attention being called to the fact that the drawings are primarily for illustration, and should not be regarded as limiting.

### BRIEF DESCRIPTION OF THE DRAWINGS

Thus, the objects, features, and advantages of the present invention are more fully understood when considered in conjunction with the following accompanying drawings:

FIG. 1 presents schematics of microstructures of cellular solids with (a) closed-cell topology, (b) partially open-cell topology, and (c) open-cell topology;

FIG. 2 is a schematic of a method and an apparatus for manufacturing cellular solids according to this invention;

FIG. 3 is another schematic of a method and an apparatus for manufacturing cellular solids according to this invention;

FIG. 4 is a further schematic of a method and an apparatus for manufacturing cellular solids according to this invention;

FIG. 5 presents another method and apparatus for manufacturing cellular solids according to a specific embodiment of the invention herein;

FIG. 6 illustrates a section of a rotating screw or conveying element inside a stationary, hollow housing;

FIG. 7 shows schematics of melt flow in the extrusion channel: (a) without gas bubbles in the melt and (b) with gas bubbles in the melt when the gas pulse valve or on/off valve is off;

FIG. 8 depicts a schematic of gas flow into the extrusion channel through an internally hollow needle connected to an on/off valve and a gas reservoir.

FIG. 9 presents approximations of the gas- and melt flow rates in the extrusion channel at a feeding port for injecting cell-fluid: (a) gas flow rate versus time, and (b) melt flow rate versus time.

FIG. 10 schematically shows foam formation in a mold with (a) small volume fraction of gas bubbles (e.g., cells) and (b) large volume fraction of gas.

FIG. 11 schematically presents the cooling and solidification process of a plasticized foam in a mold.

FIG. 12 presents non-limiting schematics of cell-fluid feeding units attached to the housing.

FIG. 13 presents further non-limiting schematics of cell-fluid feeding units attached to the housing.

FIG. 14 is a photograph of an apparatus for the continuous manufacture of cellular solids.

FIG. 15 shows the fluid structures in the extrusion channel to produce minimally-porous and cellular solids with volume fraction of voids of (a) 0.01, (b) 0.26, (c) 0.51, and (d) 0.72.

FIG. 16 presents images of the effluent (e.g., the plasticized matrix or foam) from the extrusion channel exit port while producing minimally-porous and cellular solids with volume fraction of voids of (e) 0.01, (f) 0.26, (g) 0.51, and (h) 0.72.

FIG. 17 shows scanning electron micrographs of the resulting microstructures of minimally-porous and cellular solids with volume fraction of voids of (i) 0.01, (k) 0.26, (l) 0.51, and (m) 0.72.

FIG. 18 presents measured versus calculated volume fractions of voids in the example minimally-porous and cellular solids produced.

FIG. 19 shows the drug release performance of the experimental dosage forms produced (a) fraction of drug dissolved versus time, and (b) measured value of t_{0.8} versus volume fraction of voids.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises” are not intended to exclude other additives, components, integers or steps. As used in this application, the terms “about”, “approximately” and “roughly” are used as equivalents. Any numerals used in this application with or without about/approximately/roughly are meant to cover any normal fluctuations appreciated by one of ordinary skill in the relevant art.

In the context of the invention herein, a solid is a sample of matter that retains its shape and structure when not confined. This includes the highly viscous or visco-elastic semi-solids, which in some cases may change their shape slowly under the application of their own weight. Thus in this invention, a sample of matter may be considered solid if its maximum shear viscosity is greater than 5,000 Pa·s at a shear rate no greater than 0.1 l/s. This includes but is not limited to a maximum shear viscosity greater than 10,000 Pa·s, or greater than 20,000 Pa·s, or greater than 30,000 Pa·s, or greater than 40,000 Pa·s, or greater than 60,000 Pa·s, or greater than 80,000 Pa·s, or greater than 100,000 Pa·s, or greater than 200,000 Pa·s, or greater than 500,000 Pa·s at a shear rate no greater than 0.1 l/s.

A granular solid in this disclosure is a conglomeration of discrete solid particles of a specific composition. The average size of the particulates (e.g., the average diameter or the third root of the average volume) may be in the range from about 0.01 μm or smaller to about 100 mm or greater. This includes, but is not limited to an average size of the particulates of 1 μm-100 mm, 1 μm-50 mm, 5 μm-50 mm, 10 μm-50 mm, or 5 μm-30 mm.

In the context of the invention herein, a plasticized matrix is a viscous material comprising a minimum shear viscosity of 0.005-5000,000 Pa·s at a shear rate no greater than 10 l/s. This includes but is not limited to a minimum shear viscosity of 0.01-5000,000 Pa·s, 0.025-1000,000 Pa·s, 0.05-500,000 Pa·s, 0.1-200,000 Pa·s, 0.25-100,000 Pa·s, 0.25-500,000 Pa·s, 0.5-200,000 Pa·s, 1-200,000 Pa·s, 1-500,000 Pa·s, 1-100,000 Pa·s, or 1-50,000 Pa·s at a shear rate no greater than 10 l/s. Non-limiting examples of a plasticized matrix include but are not limited to polymer melts, concentrated solutions of one or more polymers and one or more solvents (e.g., water, ethanol, acetone, isopropanol, etc.), suspensions of solid particulates or granules and a polymer melt, or suspensions of solid particulates and a concentrated polymeric solution, etc. It may be noted that in the context of the invention herein the terms “plasticized matrix”, “plasticized material”, and “melt” are used interchangeably.

A plasticized foam in the context of this disclosure is a two-phase material comprising a plasticized matrix as one phase and one or more cells (e.g., one or more bubbles or one or more drops, etc.) filled with cell-fluid as the other phase. Typically, the volume fraction of cells in a representative control volume of the plasticized foam is between 0.05 and 0.98. This includes, but is not limited to a volume fraction of cells with respect to a representative volume of the plasticized foam of 0.05-0.95, 0.1-0.9, 0.15-0.9, or 0.15-0.85. It may be noted that in some non-limiting embodiments, the cell-fluid may solidify after the plasticized foam is formed. All such two-phase materials comprising a plasticized matrix and a solidifying cell-fluid are referred to in this disclosure as “plasticized foam”.

In the invention herein, a liquid is a substance that flows freely and is not or only minimally compressible. As used herein, a sample of matter may be considered to flow freely if its shear viscosity is no greater than 5000 Pa·s in the shear rate range 1-100 l/s. This includes but is not limited to a shear viscosity no greater than 2000 Pa·s, or no greater than 1000 Pa·s, or no greater than 500 Pa·s, or no greater than 200 Pa·s, or no greater than 100 Pa·s, or no greater than 50 Pa·s, or no greater than 20 Pa·s, or no greater than 5 Pa·s, or no greater than 1 Pa·s, or no greater than 0.5 Pa·s in the shear rate range 1-100 l/s. Also, in this invention, a sample of matter may be considered not or only minimally compressible if the change in volume of said matter is no more than 5 percent of its average volume upon changing the pressure of the matter from 0.1 to 1 MPa. Non-limiting examples of samples of matter that are liquid at room temperature include water, milk, formic acid, acetic acid, polyethylene glycol 300, Poloxamer 124, 2-Pyrrolidone, Glycerol triacetate (Triacetin), D-alpha tocopheryl polyethylene glycol 1000 succinate (TPGS), Polyoxyl Hydroxystearate, Polyoxyl 15 Hydroxystearate, Castor oil, Polyoxyl castor oil (Polyethoxylated castor oil), Polyoxyl 35 castor oil, Polyoxyl hydrogenated castor oil, Glyceryl monooeleate, Glycerin, Propylene glycol, Propylene carbonate, Propionic acid, Peanut oil, Sesame oil, Olive oil, Almond oil, or combinations of such liquids with a polymer or any other molecule that dissolves in them, among others.

A gas in this invention is a substance which expands freely to fill any space available. A gas is further compressible. Non-limiting examples of gases are air, nitrogen, oxygen, CO₂, argon, helium, etc.

In this invention, a cell-fluid refers to a liquid or a gas (or combinations thereof) for injection into a plasticized matrix. As used herein, a solvent refers to a substance that dissolves a solute, resulting in a solution. A solvent is usually a liquid. Non-limiting examples of solvents include water, ethanol, acetone, isopropanol, dimethylsulfoxide, acetonitrile, ethyl acetate, toluene, hexane, pentane, benzene, cyclohexane, chloroform, diethyl ether, dichloromethane, dimethylformamide, nitromethane, formic acid, methanol, acetic acid, or n-butanol, among others.

In this disclosure, a thermoplastic material, also referred to as “thermoplastic solid” is a material which melts at a temperature lower than the temperature at which the material degrades excessively. Thus, a thermoplastic solid can be melted so that it forms a melt or plasticized matrix by heating to a temperature above its melting temperature, and then the melt can be solidified by cooling to below its melting temperature.

Moreover, in the disclosure herein, the terms “one or more active ingredients” and “drug” are used interchangeably. As used herein, an “active ingredient” or “active agent” or “active pharmaceutical ingredient” refers to an agent whose presence or level correlates with elevated level or activity of a target, as compared with that observed absent the agent (or with the agent at a different level). In some embodiments, an active ingredient is one whose presence or level correlates with a target level or activity that is comparable to or greater than a particular reference level or activity (e.g., that observed under appropriate reference conditions, such as presence of a known active agent, e.g., a positive control).

Finally, in the context of the invention herein, a three dimensional structural network of walls and edges is a structure of walls and edges that extends over a length, width, and thickness greater than 200 μm. This includes, but is not limited to structures that extend over a length, width, and thickness greater than 300 μm, or greater than 500 μm, or greater than 0.7 mm, or greater than 1 mm, or greater than 1.5 mm, or greater than 2 mm.

### DETAILED DESCRIPTION OF THE INVENTION

### Cellular Solids

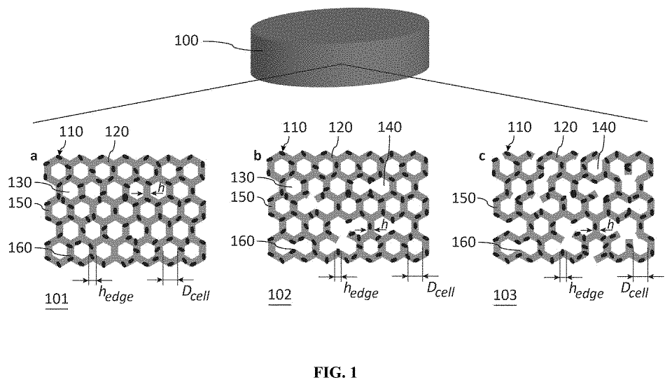

As shown in the non-limiting schematics of FIG. 1, a cellular solid 100 consist of one or more solid or semi-solid constituents 110, 120, also referred to here as “solid material”, and one or more pockets or cells 130, 140, also referred to here as “cells” or “voids”. The cells are filled with matter that can be in the gas, liquid, or solid state. The cells are closed 130 if the solid material 110, 120 is distributed in thin walls 150 that form the faces of the cells; they can be interconnected, or open 140, if some or all the walls are removed and the solid material is distributed in the cell edges 160 only. Thus FIGS. 1a, 1b, and 1c present non-limiting examples of closed-cell 101, partially open-cell 102 and open-cell structures 103. The cellular structures range from near-perfectly ordered, regular three-dimensional frameworks (as, for example, a honeycomb structure) to three-dimensional networks of the solid material with random (or almost random) distribution of the cells. The cell shape may be elliptical, polygonal, or combinations thereof, among others. Also, the cell size (e.g., the diameter of cells, D_{cell}), or the wall thickness, h, or the edge thickness, h_{edge}, of the solid material (e.g., the “thickness” of the solid material within the cellular structure) may be uniform or non-uniform.

Further non-limiting examples of cellular structures can be found in the commonly owned references M. F. Ashby, “The mechanical properties of cellular solids”, Metall. Trans. A, 14A (1983) 1755-1769; L. J. Gibson, M. F. Ashby, “Cellular solids: structure and properties”, second edition, Cambridge University Press, 1999; L. J. Gibson, M. F. Ashby, “Cellular solids in medicine”, second edition, Cambridge University Press, 1999.

Any more examples of cellular solids or structures would be obvious to a person of ordinary skill in the art. All of them are within the scope of this invention.

### Compositions

The compositions of the solid or semi-solid constituents (e.g., the solid or semi-solid walls or edges of the cellular solid and/or the content of the cells if the cells comprise solid matter) are determined by the specific application of the cellular structure. They include, but are not limited to organic materials, such as one of pharmaceutical materials (e.g., active ingredients and/or excipients (e.g., polyethylene glycol (PEG), polyethylene oxide, polyvinylpyrrolidone (PVP), PEG-PVP copolymer, poloxamer, lauroyl macrogol-32 glycerides, polyvinylalcohol (PVA), PEG-PVA copolymer, polylactic acid, polyvinylacetate phthalate, polymethacrylates (e.g., poly(methacrylic acid, ethyl acrylate) 1:1, or butylmethacrylat-(2-dimethylaminoethyl)methacrylat-methylmathacrylat-copolymer), gelatin, cellulose or cellulose derivatives (e.g., microcrystalline cellulose, hydroxypropyl cellulose, hydroxyethyl cellulose, methyl cellulose, hydroxypropyl methyl ether cellulose, hydroxypropyl methylcellulose, etc.), starch, polylactide-co-glycolide, polyvinyl caprolactam-polyvinyl acetate-polyethylene glycol graft copolymer, lactose, starch derivatives (e.g., pregelatinized starch or sodium starch glycolate, etc.), chitosan, pectin, polyols (e.g., lactitol, maltitol, mannitol, isomalt, etc.), acrylic acid crosslinked with allyl sucrose or allyl pentaerythritol (e.g., carbopol) or polyacrylic acid, among others)), foods or food-like materials (e.g., starch (e.g., potato starch, rice starch, corn starch, pregelatinized starch, etc.), amylose, amylopectin, polysaccharides, chocolate liquor, cocoa butter, cocoa, cocoa paste, fat, carbohydrates, lipids, proteins, vitamins etc.), sweeteners (e.g., sugars or polyols (e.g. glucose, sucrose, mannitol, maltitol, sorbitol, maltodextrin, xylitol, etc.)), polymers (e.g., polyethylene, polypropylene, polystyrene, polycarbonate. acrylonitrile butadiene styrene, etc.) proteins (e.g., collagen, glutelin, etc.), as well as inorganic materials, such as metals (e.g., iron, aluminum, steel, stainless steel, copper, iridium, platinum, tungsten, etc.), or ceramics.

If the content of the cells comprises a gas, the composition of the cells may include, but is not limited to one of air, oxygen, argon, nitrogen, CO₂, nitric oxide, helium, and so on. Similarly, if the content of the cells comprises a liquid, the composition of the cells may include, but is not limited to one of water, milk, olive oil, etc.

Any more examples of the compositions of the one or more solid or semi-solid walls or edges and the cells of the cellular solids would be obvious to a person of ordinary skill in the art. All of them are within the scope of this invention.

### Aspects of the Method

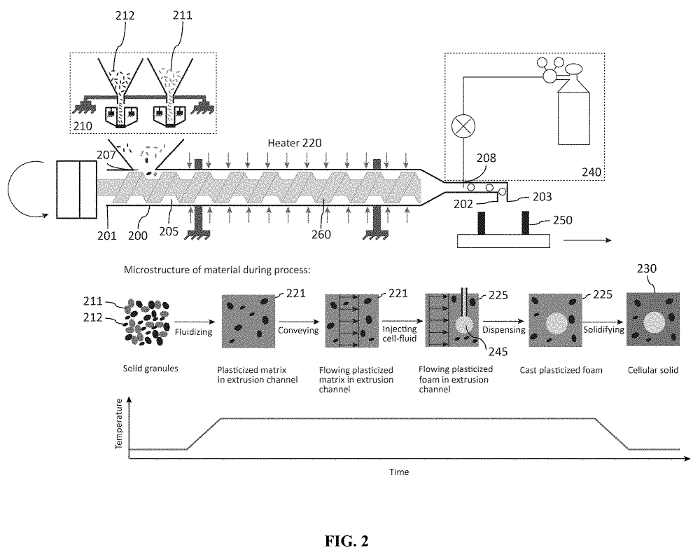

FIG. 2 presents a non-limiting example of a method to manufacture a cellular solid according to this invention. One or more granular solids 210, 215 are first injected into an extrusion channel 205 having a cross section extending along its length inside a housing 200. The injected one or more granular solids 211, 212 are then heated to a temperature greater than the melting temperature of at least one injected granular solid 211. Thus at least one injected granular solid 211 is fluidized (i.e., it transitions from solid or solid-like to fluidic or fluid-like) upon heating. The volume fraction of the fluidized granular solids 211 with respect to the volume of the one or more injected granular solids 211, 212 is so that the one or more injected granular solids 211, 212 form a plasticized matrix 221 upon heating. The plasticized matrix 221 is then conveyed towards an exit port 203 of the extrusion channel 205 by applying mechanical work on the plasticized matrix 221 along the extrusion channel 205. As the plasticized matrix 221 transits towards the exit port 203, cell-fluid 245 is injected into the extrusion channel 205 to charge the plasticized matrix 221 with cell-fluid 245 and form a plasticized foam 225. The plasticized foam 225 is then dispensed through an exit port 203 into a mold 250 or on a surface. The temperature of the dispensed plasticized foam 225 may then be reduced to below the melting temperature of the plasticized foam 225 to solidify the plasticized foam 225 and form a cellular solid 230.

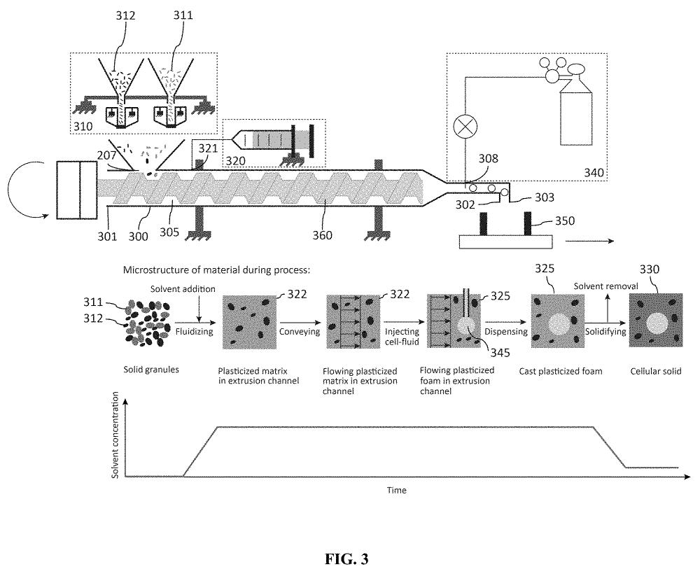

FIG. 3 shows another non-limiting example of a method to manufacture a cellular solid according to this invention. One or more granular solids 311, 312 are first injected into an extrusion channel 305 having a cross section extending along its length inside a housing 300. Then a solvent is injected into the extrusion channel 305 to solvate at least one granular solid 311. The amount of solvent injected and the volume fraction of the solvated granular solids with respect to the volume of the one or more injected granular solids 311, 312 are so that the one or more injected granular solids 311, 312 form a plasticized matrix 321 upon solvent injection. The plasticized matrix 321 is then conveyed towards an exit port 303 of the extrusion channel 305 by applying mechanical work on the plasticized matrix 321 along the extrusion channel 305. As the plasticized matrix 321 transits towards the exit port, cell-fluid 345 is injected into the extrusion channel 305 to charge the plasticized matrix 321 with cell-fluid 345 and form a plasticized foam 325. The plasticized foam 325 is then dispensed through an exit port 303 into a mold 350 or on a surface. In some embodiments, the concentration of solvent in the dispensed plasticized foam 325 may subsequently be reduced to solidify the cellular structure.

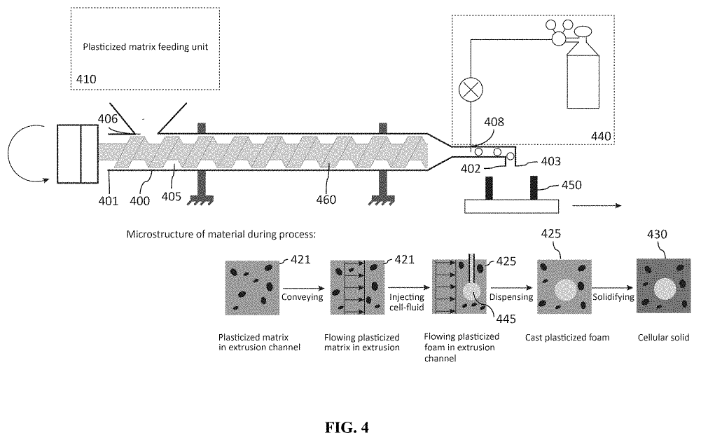

FIG. 4 is a further non-limiting example of a method of manufacturing cellular solids according to the invention herein. A plasticized matrix 421 is injected into an extrusion channel 405 having a cross section extending along its length inside a housing 400. The plasticized matrix 421 is then conveyed towards an exit port 403 of the extrusion channel 405 by applying mechanical work on the plasticized matrix 421 along the extrusion channel 405. As the plasticized matrix 421 transits towards the exit port 403, cell-fluid 445 is injected into the extrusion channel 405 to charge the plasticized matrix 421 with cell-fluid 445 and form a plasticized foam 425. The plasticized foam 425 is then dispensed through an exit port 403 into a mold 450. In some embodiments, the dispensed plasticized foam 425 may subsequently be solidified.

It may be obvious to a person of ordinary skill in the art that in any example presented herein, the extrusion channel may comprise one or multiple exit ports for dispensing a plasticized foam into one or multiple molds or one or multiple surfaces. Also, the one or more exit ports or the one or more molds or the one or more surfaces may move horizontally or vertically as the plasticized foam is dispensed into a mold. Furthermore, the extrusion channel may be equipped with elements to control the mass or volume of plasticized foam dispensed, such as piezo-electric printheads, fluid valves (e.g., needle valves, etc.), injection pistons, or cutters to cut the stream of plasticized foam effluent from an exit port, among others. Moreover, sensors may be attached to the extrusion channel or mold. Such sensors include, but are not limited to pressure sensors, flow meters, or balances for measuring the weight of the dispensed plasticized foam, among others.

Any more examples of the process steps to manufacture the cellular solids would be obvious to a person of ordinary skill in the art. All of them are within the scope of this invention.

### Aspects of the Apparatus

FIG. 2 also presents a non-limiting schematic of an apparatus to manufacture the cellular solids according to this invention. The apparatus comprises an internally hollow housing 200 having an internal surface encapsulating and defining an extrusion channel 205 having a first end 201 and a second end 202 and a cross section extending axially along its length from said first end 201 to said second end 202 and terminating into an exit port 203 at the second end 202. The housing 200 has at least a first feeding port 207 between the first end 201 and second end 202 for injecting at least one granular solid 211, 212 into the extrusion channel 205 and has at least a second feeding port 208 between the first feeding port 207 and the exit port 203 for injecting cell-fluid 245 into the extrusion channel 205. The apparatus further comprises at least one heating element 220 for fluidizing at least one injected granular solid to form a plasticized matrix 221 in the extrusion channel 205. The apparatus further comprises a conveying element 260 for extruding the plasticized foam 225 in the extrusion channel 205 through an exit port 203, and a mold 250 or a surface (e.g., a stage, such as linear or rotary stage, etc.) for accepting the extruded plasticized foam 225. In some embodiments, the plasticized foam solidifies in the mold 250. The mold may further comprise an element for ejecting the cellular solid 230 from the mold 250 or from the surface. Furthermore, in some embodiments the apparatus further comprises a granular solid feeding unit 210 for injecting at least one granular solid 211, 212 through the first feeding port 207 into the extrusion channel 205. In some embodiments, moreover, the apparatus further comprises a cell-fluid feeding unit 240 attached to the second feeding port 208 for injecting cell-fluid 245 into the plasticized matrix 221 to form a plasticized foam 225 in the extrusion channel 205.

Another non-limiting schematic of an apparatus to manufacture the cellular solids according to this invention is presented in FIG. 3. The apparatus comprises an internally hollow housing 300 having an internal surface encapsulating and defining an extrusion channel 305 having a first end 301 and a second end 302 and a cross section extending axially along its length from said first end 301 to said second end 302 and terminating into an exit port 303 at the second end 302. The housing 300 has at least a first feeding port 306 between the first end 301 and the second end 302 for injecting at least one granular solid 311, 312 into the extrusion channel 305. The housing 300 further has at least a second feeding port 307 between the first feeding port 306 and the exit port 303 for injecting at least one solvent into the extrusion channel 305 to form a plasticized matrix 321 by solvating at least one injected granular solid 311, 312. The housing 300 further has at least one third feeding port 308 between the second feeding port 307 and the exit port 303 for injecting cell-fluid 345 into the plasticized matrix 321 to form a plasticized foam 325 in the extrusion channel 305. The apparatus further comprises a conveying element 360 for extruding the plasticized foam 225 in the extrusion channel through the exit port 303, and a mold 350 or a surface (e.g., a stage, such as linear or rotary stage, etc.) for accepting the extruded plasticized foam 325. Furthermore, in some embodiments the apparatus further comprises a granular solid feeding unit 310 for injecting at least one granular solid 311, 312 through the first feeding port 306 into the extrusion channel 305. In some embodiments, moreover, the apparatus further comprises a solvent feeding unit 320 attached to the second feeding port 307 for injecting at least one solvent into the extrusion channel 305. In addition, in some embodiments the apparatus further comprises a cell-fluid feeding unit 340 attached to the third feeding port 308 for injecting cell-fluid 345 into the plasticized matrix 321 to form a plasticized foam 325 in the extrusion channel 305.

FIG. 4 presents another non-limiting schematic of an apparatus to manufacture the cellular solids according to this invention. The apparatus comprises an internally hollow housing 400 having an internal surface encapsulating and defining an extrusion channel 405 having a first end 401 and a second end 402 and a cross section extending axially along its length from said first end 401 to said second end 402 and terminating into an exit port 403 at the second end 402. The housing 400 has at least a first feeding port 406 between the first end 401 and the second end 402 for injecting a plasticized matrix 421 into the extrusion channel 405 and at least a second feeding port 408 between the first feeding port 406 and the exit port 403 for injecting cell-fluid 445 into the extrusion channel 405. The apparatus further comprises a conveying element 460 for extruding the plasticized foam 425 in the extrusion channel 405 through the exit port 403, and a mold 450 or a surface (e.g., a stage, such as linear or rotary stage, etc.) for accepting the extruded plasticized foam 425. Furthermore, in some embodiments the apparatus further comprises a plasticized matrix feeding unit 410 for injecting the plasticized matrix 421 through the first feeding port 406 into the extrusion channel 405. In some embodiments, moreover, the apparatus further comprises a cell-fluid feeding unit 440 attached to the third feeding port 408 for injecting cell-fluid 445 into the plasticized matrix 421 to form a plasticized foam 425 in the extrusion channel 405.

In any example presented herein, the extrusion channel may comprise one or multiple exit ports to dispense a plasticized foam into one or multiple molds or on one or multiple surfaces. Furthermore, the extrusion channel may be equipped with elements to control the mass or volume of plasticized foam dispensed, such as piezo-electric printheads, fluid valves (e.g., needle valves, etc.), injection pistons, or cutters to cut the stream of plasticized foam effluent from an exit port, among others. Moreover, sensors may be attached to the extrusion channel or the mold. Such sensors include, but are not limited to pressure sensors, flow meters, or balances for measuring the weight of the dispensed plasticized foam, among others.

### Process Models

The following examples present ways by which specific, non-limiting examples of the process disclosed may be modeled. The models will enable one of skill in the art to more readily understand the invention and its features. The models and examples are presented by way of illustration, and are not meant to be limiting in any way.

### (a) Process Overview

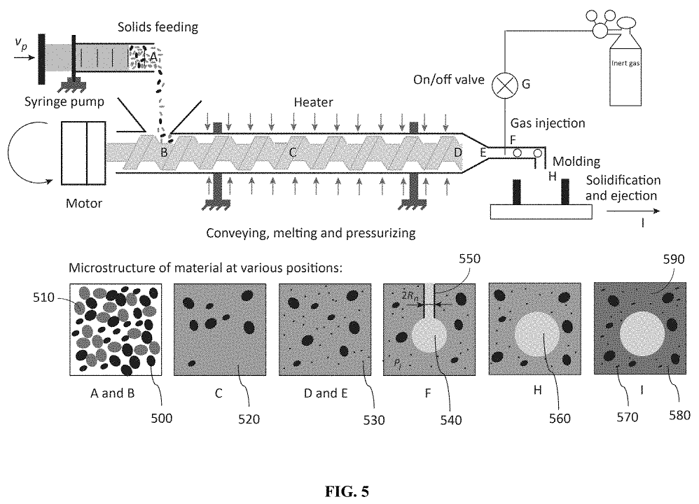

The models presented refer mostly to the non-limiting apparatus and method illustrated schematically in FIG. 5. FIG. 5 resembles the design and conditions of the apparatus and method applied for preparing the experimental examples of cellular solids shown later.

As shown in FIG. 5, the input material is a mixture of one or more granular solids, which are filled in a syringe with uniform barrel diameter at point A. By controlled displacement of the syringe's piston the powder mixture (e.g., the one or more granular solids) is fed through a hopper into an extrusion channel at B. In the extrusion channel, multiple unit steps are integrated into a continuous process. First the solid granules are conveyed away from the inlet by the rotating screw. Additionally, the one or more granular solids are mixed further as they are conveyed along the screw. Furthermore, because the barrel temperature (e.g., the temperature of the housing) is kept above the melting temperature of at least one injected granular solid, said at least one injected granular solid melts and plasticizes the mixture as it is transported forward in section BC. The mixture is plasticized (e.g., the one or more granular solids are converted to a plasticized matrix or melt) at point C. In section CD the pressure of the melt increases as it moves forward. The high pressure generated by the extruder screw pushes the melt first through a converging section of the extrusion channel from D to E, and then through a channel section with constant diameter into which an internally hollow needle is inserted at F. The internally hollow needle is connected to an electrical shut-off valve (e.g., an on/off valve) and a high-pressure gas tank equipped with a pressure regulator. By controlling the gas pressure and the opening and closing times of the valve (i.e., the pulse width and repetition rate), a train of bubbles of well-controlled size and spacing is introduced into the fluid stream (e.g., the plasticized matrix) at F. The two-phase fluid stream (e.g., the plasticized foam) is subsequently dispensed in a mold at H. The plasticized foam is then shaped to form a cast plasticized foam, solidified, and ejected from the mold at I.

Also shown in FIG. 5 are non-limiting examples of microstructures of the material during the process. At points A and B, the material is a granular mixture of constituent A 500 and a thermoplastic constituent B 510. Then at point C the constituent A 500 is embedded in the fluidized constituent B 520 to form a plasticized matrix. As the material is transported forward along the screw, part of (or all of) constituent A may dissolve in the fluidized constituent B. Thus at points D and E, the plasticized material may contain dissolved constituent A molecules 530 in addition to the solid constituent A particles 500 and the fluidized constituent B 520. At point F, a gas bubble 540 is added through an internally hollow needle 550. Then as the material is pushed towards an exit port of the extrusion channel and the plasticized foam is dispensed into a mold, the pressure is reduced. Accordingly, at point H the gas bubble 560 has expanded compared with its initial volume. Then as the temperature of the material is reduced and the plasticized foam solidifies, some of the constituent A molecules may form nucleates and grow to small constituent A particles 570. Other constituent A molecules may remain as dissolved molecules 580 in the walls and edges of the cellular solid 590.

(b) Injecting Granular Solids into the Extrusion Channel

In the non-limiting example presented in FIG. 5, the rate at which the one or more granular solids are injected into the extrusion channel is controlled volumetrically. In a volumetric, continuous particulate feeder (e.g., a volumetric, continuous granular solid feeding unit), the mass flow rate, dM_{f}/dt, of solid particles (e.g., one or more granular solids) fed to the extrusion channel may be written as:

$$\frac{{dM}_{f}}{dt} = {ρ}_{{p }^{φ} {p}^{A} {p}^{v} p} \qquad ( 1 )$$

where ρₚ is the density of the solid particles, φₚ their volume fraction in the particle bed, Aₚ the cross-sectional area of the particle bed, and vₚ its translational velocity. vₚ may be controlled, for example, by the axial velocity of a piston, the rotation rate of a conveyor screw, or other methods.

The mass flow rate of particulates in volumetric feeders is typically subject to some variation because no rigorous mathematical models are available that adequately describe the behavior of granular matter. Consequently, φₚ and vₚ must typically be found from heuristic models. In some cases they are difficult to control precisely.

More precise control of the mass flow rate of particulates fed to the extrusion channel may be achievable, however, by gravimetric control. In this case, the mass flow rate of particulates injected is directly measured and controlled (e.g., the weight of the one or more granular solids supplied to the extrusion channel per unit time is continuously measured and controlled). Thus despite the difficulties in modeling the flow and behavior of granular matter, precise control of the rate at which particulates are fed to the extrusion channel is achievable.

Similarly, it may be noted that also a plasticized matrix may be injected into the extrusion channel by volumetric or gravimetric control of the flow rate.

Any more models or examples for injecting granular solids or a plasticized matrix into the extrusion channel would be obvious to a person of ordinary skill in the art. All of them are within the scope of this invention.

### (c) Fluidizing at Least One Granular Solid in the Extrusion Channel

Because the input to the non-limiting process shown in FIG. 5 is a solid material, solids handling is unavoidable at the beginning. It has been reported that in a typical extruder screw and barrel, from the point where the barrel temperature exceeds the melting temperature of a thermoplastic solid, at about 2-4 times the pitch of the screw forward, a small melt pool forms (see, e.g., C. G. Gogos, Z. Tadmor, “Principles of polymer processing”, second edition, John Wiley & Sons, 2006). Thus a thermoplastic granular solid can be readily fluidized by melting in a system as illustrated in FIG. 5.

Alternatively, if at least one of the injected granular solids transitions from solid or solid-like to fluidic or fluid-like upon contact with a solvent (e.g., water, acetone, dimethylsulfoxide, ethanol, ethyl acetate, etc.), said at least one granular solid may be fluidized by solvation or dissolution in the solvent. If the flux of the solvent molecules to the interior of a solid particle is faster than the flux of the particle's molecules into the solvent, the time to fluidize said particle by solvation, tₛₒₗᵥ, is roughly equal to the time the solvent molecules require to diffuse to the center of said particle. Thus for a solid particle of radius, Rₚ, in which diffusion of the solvent is Fickian,

$${t}_{solv} ≅ \frac{{R}_{p}^{2}}{{D}_{eff}} \qquad ( 2 )$$

where D_{eff} the effective diffusivity of the solvent in the particle. By way of example but not by way of limitation, if Rₚ=25 μm, D_{eff}=3×10¹⁰ m²/s (e.g., of the order of the diffusion coefficient of water in hydroxypropyl methyl cellulose), tₛₒₗᵥ=2.1 s. Thus for a screw rotating at 5-500 rpm, if each particle is surrounded by the solvent immediately after the solvent is added, the one or more granular solids are converted to a wet, plasticized material (e.g., a plasticized matrix) about 5×2.9/60−500×2.9/60=0.17−17 screw turns after the solvent feeding position. Thus, a granular solid may also be readily fluidized by solvation in an apparatus or method where solvent is injected into the extrusion channel (see, e.g., the non-limiting example of FIG. 3).

Any more examples or models to fluidize the one or more injected granular solids in the extrusion channel would be obvious to a person of ordinary skill in the art. All of them are within the scope of this invention.

### (d) Applying Mechanical Work on the Plasticized Matrix in the Extrusion Channel

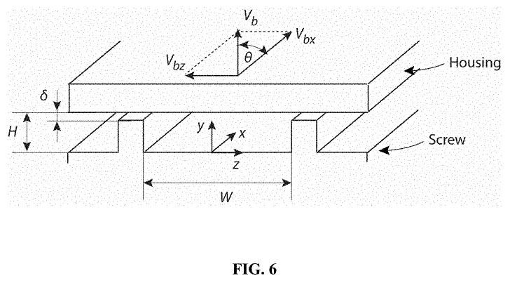

If mechanical work is applied on the plasticized matrix, it flows. In the non-limiting examples of FIGS. 5 and 6, mechanical work is applied on the plasticized matrix in the extrusion channel by a rotating extrusion screw, which transports it towards an exit port of the extrusion channel. The melt flow rate in the x-direction in the extrusion channel, or throughput of the extruder, Q, may be approximated by:

Q=Q_{d}+Qₚ (3)

where Q_{d} is the drag- and Qₚ the pressure-driven flow contribution. The drag or Couette flow, Q_{d}, is due to the rotation of the housing relative to the screw (or vice versa) and drives the material forward. It may be written as follows:

$${Q}_{d} = \frac{{V}_{bx} WH}{2} = \frac{2 π {R}_{ba} {N}_{ba} cos θ WH}{2} \qquad ( 4 )$$

where V_{bx} is the velocity of the housing relative to the screw in the x-direction, W the width of the screw channel, H the height of the screw channel, R_{ba} the radius of the extrusion channel (e.g., the internal radius of the internally hollow housing), N_{ba} the rotation rate of the housing relative to the screw, and θ the helix angle of the screw.

The pressure-driven flow rate, Qₚ, drives the material backward. If the plasticized matrix is Newtonian viscous and the flow laminar,

$${Q}_{p} = - \frac{{WH}^{3}}{12 {μ}_{m}} \frac{{P}_{D} - {P}_{C}}{L} \qquad ( 5 )$$

where μm is the viscosity of the plasticized matrix and L the length of the screw channel in the x-direction that is completely filled with the plasticized material.

Thus if, by way of example but not by way of limitation, if R_{ba}=5 mm, θ=17°, W=10 mm, and H=1 mm, the drag flow, Q_{d}=13-1252 mm³/s for N_{ba}=5-500 rpm. Under these conditions, if the pressure at C, Pc, is atmospheric, L=25 cm and μₘ=115 Pa·s, the pressure at D, P_{D}, is between atmospheric and 3.9 MPa. Thus in this non-limiting example, a variety of throughputs (or flow rates) and a variety of fluid pressures can be generated by the application of mechanical work.

It may be noted that mechanical work may alternatively be applied by the axial displacement of a piston in contact with the plasticized matrix, a peristaltic pump, or pressurized gas or liquid, among others.

Any more examples of models to apply mechanical work on the plasticized matrix in the extrusion channel so as to convey it towards an exit port would be obvious to a person of ordinary skill in the art. All of them are within the scope of this invention.

### (e) Melt Pressure in the Extrusion Channel at a Feeding Port for Injecting Cell-Fluid

If the cross section of the extrusion channel tapers down before an exit port to the cross section of said exit port, as in the non-limiting example of FIG. 5, the pressure of the plasticized matrix may be greatest at position D at the end of the extruder screw. Then the pressure decreases as the material is transported forward into the rear section of the extrusion channel and towards an exit port.

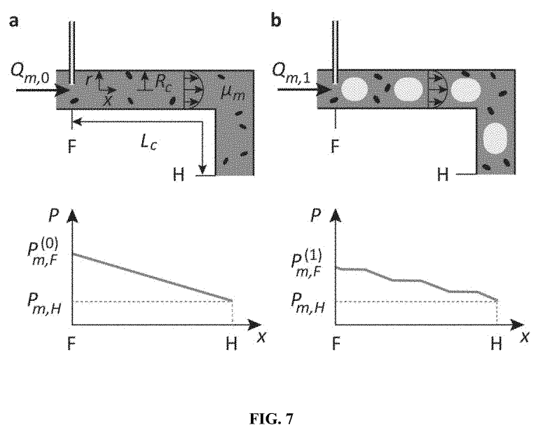

FIG. 7 presents a non-limiting example of the rear section of the extrusion channel connected to a gas injection needle at position F (a feeding port for injecting cell-fluid into the extrusion channel). The gas injection needle is connected to a pulse valve (e.g., an on/off valve) and a gas reservoir equipped with a gas pressure regulator.

In FIG. 7a, the gas pulse valve is off, i.e., no gas is injected, and the channel is filled with a Newtonian viscous, incompressible melt (also referred to here as “plasticized matrix”) without any gas bubbles. In laminar flow, the pressure drop from position F to H may be expressed by the Hagen-Poiseouille equation as:

$${P}_{m , F}^{( 0 )} - {P}_{m , H} = \frac{8 {μ}_{m} {L}_{c} {Q}_{m , 0}}{π {R}_{c}^{4}} \qquad ( 6 )$$

where P_{m,F}⁽⁰⁾ is the melt pressure at point F, P_{m,H} the pressure at H, μm the viscosity of the melt, L_{c} the length of the channel from F to H, R_{c} the channel radius, and Q_{m,0} the volumetric flow rate of the melt. Thus under the conditions of the non-limiting experimental examples 1, 2 and 3 shown later (μm=115 Pa·s, L_{c}=15 mm, Q_{m,0}=1.5 mm³/s, =500 μm, and P_{m,H}=Pₐₜₘ), by Eq. (6) P_{m,F}⁽⁰⁾=0.206 MPa. Also, the average velocity of the melt in the channel, v_{m,0}=Q_{m,0}/πR_{c}²=1.9 mm/s. Thus the Reynolds number, Re=2ρₘv_{m,0}/μm=2ρₘQ_{m,0}/πR_{c}²μₘ=4×10⁻⁵ and the Capillary number, Ca=ρₘv_{m,0/γ}=ρₘQ_{m,0}/πR_{c}²γ=4.9 (if the density of the melt, ρₘ=1150 kg/m³ and the gas-melt interfacial tension, γ=0.045 N/m). The inertial and capillary forces are therefore negligible small compared with the viscous forces in the non-limiting example shown.

In FIG. 7b, the gas pulse valve is again off, but gas bubbles of appreciable volume fraction are present in the channel. Because the viscosity of the melt is much greater than that of the gas, the pressure gradient is much greater in the melt-filled segments than in the gas-filled segments. Thus the flow resistance offered by the bubbles to overall flow may be neglected.

Assuming that the melt flow rate in a melt-filled channel segment, Q_{m,s}, is roughly the same as the melt flow rate into the two-phase channel at point F, Q_{m,1}, the pressure drop in the channel, by adapting the Hagen-Poiseouille equation, may be written as:

$${P}_{m , F}^{( 1 )} - {P}_{m , H} = \frac{8 {μ}_{m} {L}_{m} {Q}_{m , 1}}{π {R}_{c}^{4}} \qquad ( 7 )$$

where P_{m,F}⁽¹⁾ is the melt pressure at point F of the two-phase channel and Lₘ the length of the melt-filled fraction of the channel. Lₘ may be written in terms of the volume fraction of melt in the channel, φ_{m,c}, and the total channel length, L_{c}, as

Lₘ=ϕ_{m,C}L_{c} (8)

Thus if Q_{m,1} is 1.03-1.15 times greater than Q_{m,0} and φ_{m,c} is between about 0.31 and 0.83, by Eqs. (7) and (8) P_{m,F}⁽¹⁾ is in the range 0.139-0.191 MPa when the gas valve is closed but gas bubbles are present in the channel (as listed in Table 1, later).

Any more examples or models to determine the melt pressure in the extrusion channel at a feeding port for injecting cell-fluid would be obvious to a person of ordinary skill in the art. All of them are within the scope of this invention.

(f) Gas Injection into the Plasticized Matrix in the Extrusion Channel

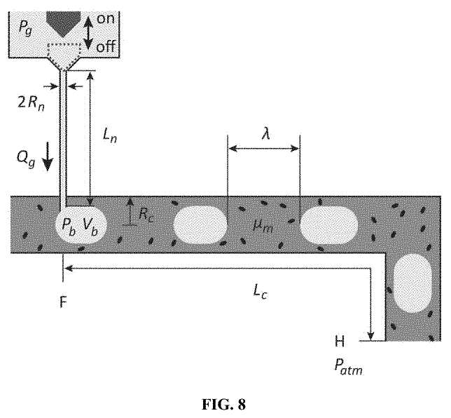

To inject gas bubbles (or liquid droplets) into the melt stream in a system as shown in FIG. 8, the bubble pressure, P_{b}, at the needle channel exit port should be greater than the sum of the melt pressure at F, P_{m,F}, and the capillary pressure, P_{cap}. Thus

$${P}_{b} > {P}_{m , F} + {P}_{cap} = {P}_{m , F} + \frac{2 γ}{{R}_{n}} \qquad ( 9 )$$

where γ is the tension of the gas-fluid interface and Rₙ the inner radius of the microfluidic channel at the needle channel exit port. Under the conditions of the non-limiting experimental examples 1, 2 and 3 shown later (Rₙ=25 μm and γ=0.045 N/m), P_{cap}=3.6 kPa. This is much smaller than the values of P_{m,F} calculated above. Thus gas may be injected into the melt at about P_{m,F} in this case. (Note that P_{m,F}≈P_{m,F}⁽⁰⁾ for a melt-filled channel without gas bubbles and P_{m,F}≈P_{m,F}⁽¹⁾ for a channel with gas bubbles).

If the gas pressure is only slightly greater than the melt pressure, very small gas bubbles will be released slowly from the internally hollow needle into the flowing melt stream. If the object, however, is to create structures with considerable volume fraction of cells or voids, the gas pressure in the bubble during gas injection must be considerably greater than the melt pressure. In that case, melt flow into the extrusion channel may be virtually blocked as the gas flows into the channel.

In the non-limiting schematic of FIG. 8, gas flows through an internally hollow needle into an extrusion channel. During gas injection, melt flow at position F is essentially stopped. Neglecting the flow resistance of the gas-filled regions in the extrusion channel and the internally hollow needle, and assuming that the pressure gradient is the same in each of the melt-filled segments, the bubble expansion rate, dV_{b}/dt, may be expressed as follows:

$${Q}_{g} = \frac{{dV}_{b}}{dt} = \frac{π {R}_{c}^{4} ( {P}_{g} - {P}_{atm} )}{8 {μ}_{m} {L}_{m}} \qquad ( 10 )$$

where P_{g} is the gas pressure set by the regulator connected to the gas reservoir. Under the conditions of the non-limiting experimental examples 1, 2 and 3 shown later, the gas pressure is constant at P_{g}=0.35 MPa, thus Q_{g} is between 4.27 and 11.35 mm³/s if Lₘ/L_{c}=φ_{m,c}=0.31-0.83. Thus gas flow rates of the order of or greater than the melt flow rates are readily obtained and Q_{g} is readily controlled by the gas injection pressure, the geometry of the extrusion channel and internally hollow needle, and the viscosity of the plasticized matrix or melt.

Any more examples or models of gas (or liquid) injection would be obvious to a person of ordinary skill in the art. All of them are within the scope of this invention.

### (g) Microstructure of the Plasticized Foam in the Extrusion Channel

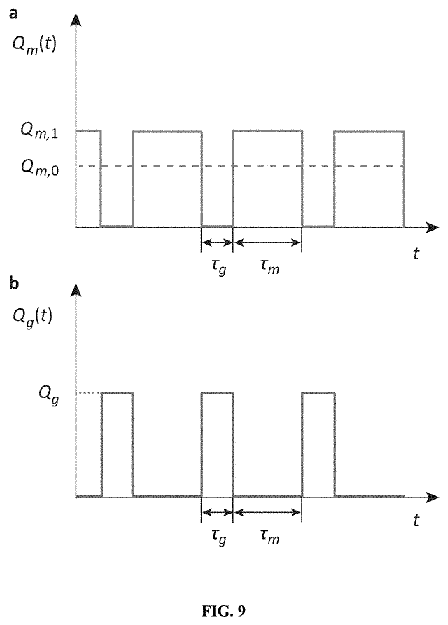

The volume fraction of melt in the extrusion channel, φ_{m,c}, (e.g., the volume fraction of melt in the plasticized foam) is equal to the ratio of melt volume to total volume in the channel. It may be estimated from the volumes of the melt- and gas-filled segments which are in turn determined by the melt- and gas flow rates. If melt flow into the channel is blocked during gas injection, and P_{m,F}⁽¹⁾ (the melt pressure at F when the valve is closed) and φ_{m,c} are assumed constant for a given set of conditions, the profile of the melt flow rate versus time at point F follows roughly a square wave as shown in FIG. 9a. The melt flow rate into the channel when the valve is closed, Q_{m,1}, may thus be derived from the time-average volumetric melt flow rate, Q_{m,0}, as:

$${Q}_{m , 1} ≅ {Q}_{m , 0} \frac{{τ}_{g} + {τ}_{m}}{{τ}_{m}} \qquad ( 11 )$$

where τ_{g} and τₘ, respectively, are the times when the valve is open and closed in a pulsing cycle (e.g., τ_{g} is the opening time and τₘ the closing time of the valve in one repetition period). For the non-limiting range of pulsing parameters, τ_{g}=20-60 ms and τₘ=400-600 ms, Q_{m,1}≅1.03-1.15×Q_{m,0}.

The volume of a melt-filled segment, Vₘ, is then determined by Q_{m,1} and the time τₘ during which melt flows into the channel in a pulsing cycle (i.e., the time when the valve is closed):

Vₘ=Q_{m,1}τₘ (12)

The melt may be assumed incompressible, and thus Vₘ is constant as the melt-filled segment flows forward along the channel. Accordingly, the length of a melt-filled segment, or the mean “free” distance between the bubbles in the channel, λ, is about:

$$λ = \frac{{Q}_{m , 1}}{π {R}_{c}^{2}} {τ}_{m} \qquad ( 13 )$$

Under the conditions of the non-limiting experimental examples 1, 2 and 3 shown later, by Eq. (13) λ=879-1184 μm (as shown in Table 1).

The gas flow rate into the channel at point F, Q_{g}, by Eq. (9) is roughly constant when the valve is open, provided that φ_{m,c} is again assumed time-invariant. When the valve is closed, the gas flow rate is zero for an internal volume of the needle (e.g., the volume of the microfluidic channel in the hollow needle) so small that the amount of compressed residual gas in it is negligible. Thus also the profile of the gas flow rate versus time into the channel roughly follows a square wave as shown in FIG. 9b. Unlike the melt-filled segments, however, the gas-filled segments may not be assumed incompressible. The volume of a gas-filled segment increases as it flows forward along the channel and the pressure is decreased. A rough estimate of the average volume of a gas-filled segment, V_{g}, in the channel is the average of the segment volumes at points F and H, respectively, and may be written as:

$${V}_{g} ≅ \frac{1}{2} ( {Q}_{g} {τ}_{g} + \frac{{P}_{g}}{{P}_{atm}} {Q}_{g} {τ}_{g} ) = \frac{{P}_{g} + {P}_{atm}}{2 {P}_{atm}} {Q}_{g} {τ}_{g} \qquad ( 14 )$$

where τ_{g} is the time during which the gas flows into the channel in a pulsing cycle (i.e., the time when the valve is open).

Now the volume fraction of melt in the channel (e.g., in the plasticized foam) can be derived. At steady-state, φ_{m,c} is about the same as the melt volume fraction in an average pair of melt- and gas-filled segments. Thus

$${φ}_{m , c} = \frac{{V}_{m}}{{V}_{m} + {V}_{g}} = \frac{{Q}_{m , 1} {τ}_{m}}{{Q}_{m , 1} {τ}_{m} + \frac{{P}_{g} + {P}_{atm}}{2 {P}_{atm}} {Q}_{g} {τ}_{g}} = {( 1 + \frac{{P}_{g} + {P}_{atm}}{2 {P}_{atm}} \frac{{Q}_{g}}{{Q}_{m , 1}} \frac{{τ}_{g}}{{τ}_{m}} )}^{- 1} \qquad ( 15 )$$

Eqns. (8), (10), (11), and (15) may also be combined to give the following expression for the bubble volume produced per pulse at point F, V_{b,F}, for an internal volume of the needle that is negligible small:

$${V}_{b , F} = \frac{{dV}_{b}}{dt} {τ}_{g} = \frac{π {R}_{c}^{4} ( {P}_{g} - {P}_{atm} ) {τ}_{g}}{8 {μ}_{m} {L}_{c}} {( 1 - \frac{{P}_{g} + {P}_{atm}}{2 {P}_{atm}} \frac{π {R}_{c}^{4} ( {P}_{g} - {P}_{atm} )}{8 {μ}_{m} {L}_{c} {Q}_{m , 0}} \frac{{τ}_{g}}{{τ}_{g} + {τ}_{m}} )}^{- 1} \qquad ( 16 )$$

Under the conditions of the non-limiting experimental examples 1, 2, and 3 shown later, by Eq. (16) V_{b,F}=0.233 mm³ if P_{g}=0.35 MPa, τ_{g}=40 ms and τₘ=500 ms (Table 1). We may note that the dimensionless term on the right hand side of Eq. (16) is equal to 1/φ_{m,c}, which by Eq. (16) is between about 1/0.31 and 1/0.83 under the conditions of the non-limiting experimental examples shown later. Thus the microstructure of the plasticized foam in the channel is readily controlled by the gas injection pressure, the melt flow rate, the geometry of the channel, and the pulsing parameters of the valve.

Any more examples or models to inject gas (or liquid) into the extrusion channel and to model the microstructure of the liquid foam in the extrusion channel would be obvious to a person of ordinary skill in the art. All of them are within the scope of this invention.

### (h) Microstructure of the Plasticized Foam at the Exit Port

At the exit port of the extrusion channel, the melt pressure is reduced and in the non-limiting experimental examples presented herein it is about equal to atmospheric pressure. Accordingly, the bubble volume at H, V_{b,H}, from Boyle's law may be written as:

$${V}_{b , H} = \frac{{P}_{g}}{{P}_{atm}} {V}_{b , F} \qquad ( 17 )$$

Assuming that the bubble or cell shape in the plasticized foam is spherical, the cell size, D_{cell}, is about:

$${D}_{cell} = {( \frac{6 {V}_{b , H}}{π} )}^{\frac{1}{3}} \qquad ( 18 )$$

Under the conditions of the non-limiting experimental examples presented, by Eq. (18) D_{cell}=833-1664 μm (see Table 1).

The volume fraction of gas (or voids) in the plasticized foam at atmospheric pressure, φᵥ, is about:

$${φ}_{v} = \frac{{P}_{g} {Q}_{g} {τ}_{g}}{{P}_{atm} {Q}_{m , 1} {τ}_{m} + {P}_{g} {Q}_{g} {τ}_{g}} = {( 1 + \frac{{P}_{atm}}{{P}_{g}} \frac{{Q}_{m , 1}}{{Q}_{g}} \frac{{τ}_{m}}{{τ}_{g}} )}^{- 1} \qquad ( 19 )$$

By combining this equation with Eqns. (8), (10), (11), and (15), an intricate term is obtained for φᵥ, which is found to be between 0.24 and 0.77 for the conditions of the non-limiting experimental examples presented (Table 1). Thus the microstructure of the plasticized foam is determined by well-controllable parameters in the system presented.

Furthermore, it may be noted that if P_{m,F}⁽¹⁾ were measured and controlled, φᵥ could be derived directly from Eqs. (7), (10), and (19) as:

$${φ}_{v} = {( 1 + \frac{{P}_{atm}}{{P}_{g}} \frac{{P}_{m , F}^{( 1 )} - {P}_{atm}}{{P}_{g} - {P}_{atm}} \frac{{τ}_{m}}{{τ}_{g}} )}^{- 1} \qquad ( 20 )$$

which underscores that there is a benefit of monitoring and controlling the melt pressure at F for achieving a plasticized foam with readily predictable microstructure.

Any more examples to model the microstructure of the plasticized foam would be obvious to a person of ordinary skill in the art. All of them are within the scope of this invention.

### (i) Mold Filling

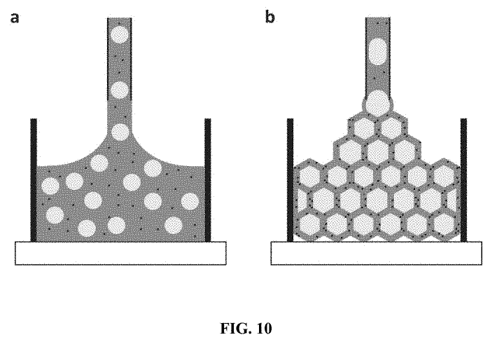

The plasticized foam may subsequently be dispensed into a mold cavity with the shape of the desired product as shown in the non-limiting example of FIG. 10. A low viscosity melt would be desirable to spread and distribute the material evenly in the mold. However, such a system also exhibits fast bubble dynamics. For example, the Hadamard-Rybczynski equation for the terminal rising bubble velocity, V_{b}, of a gas bubble with density, ρ_{g}<<ρₘ, and viscosity, ρ_{g}<<μₘ, is:

$${v}_{b} = \frac{1}{3} \frac{{D}_{cell}^{2} g {ρ}_{m}}{4 {μ}_{m}} \qquad ( 21 )$$

By Eq. (21), in a low-viscosity melt with ρₘ=1150 kg/m³ and ρₘ=2 Pa·s (e.g., PEG 8k at 70° C.), a bubble of diameter D_{cell}=800 mm assumes a terminal rising velocity of 0.3 mm/s. Thus if bubble rise must be less than about 300 μm so as to preserve the microstructure of the liquid foam (i.e., to avoid such problems as bubble assembly at the top of the dosage form, and bubble coalescence and bursting), the melt must solidify within about 1 second after discharge from the capillary channel in this non-limiting case.

The bubble rise and bursting and coalescence time can be delayed by increasing the viscosity of the melt sufficiently. Higher viscosity fluids (and fluids with large bubble content) may, however, grow vertically in the mold and not spread evenly. But this problem can be overcome by positioning the mold close to the channel exit, and by moving it in the horizontal plane.

Any more examples or models of mold filling or the microstructural evolution of the plasticized foam after mold filling would be obvious to a person of ordinary skill in the art. All of them are within the scope of this invention.

### (j) Solidification of the Plasticized Foam

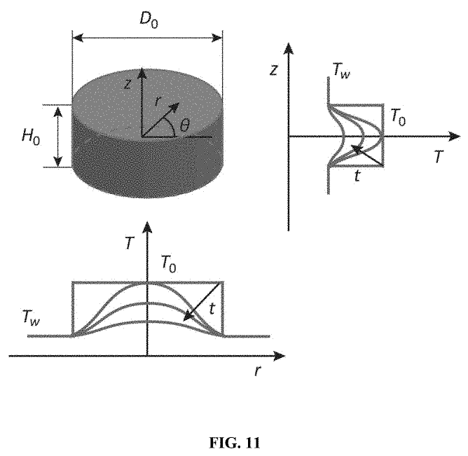

To preserve the microstructure of the plasticized foam, and to achieve high process rates, solidification of the plasticized foam should be fast. FIG. 11 presents a non-limiting example of a liquid foam that is solidified by cooling. The liquid foam of this example has a diameter greater than the thickness, i.e., D₀>H₀. The spatial derivatives of the temperature gradients are therefore greater axially than radially.

Thus the cooling time τ_{c} (i.e., the time required for the center of the plasticized foam to reach a temperature, T_{c}, which is of the order of the temperature at which the plasticized foam solidifies) may be approximated as:

$${τ}_{c} = \frac{ρ c}{k} \frac{{H}_{0}^{2}}{{π}^{2}} ln ( \frac{4}{π} \frac{{T}_{0} - {T}_{w}}{{T}_{c} - {T}_{w}} ) \qquad ( 22 )$$

where ρ is the density of the molded plasticized foam, c its specific heat, and k its thermal conductivity. Initially, the plasticized foam is at the temperature T₀. The boundary condition to Eq. (22) is that the mold wall temperature, T_{w}, is kept constant.

The molded plasticized foam is not a homogeneous material because it comprises one or more molten constituents and gas bubbles. The one or more constituents may be assumed to have the same thermal properties, but their combined thermal conductivity, specific heat, and density may be vastly different from those of the gas bubbles. Therefore, to calculate τ_{c}, it may be required to calculate the effective thermal properties of the composite medium.

The density and heat capacity are scalar properties, thus their effective values are equal to the average values

ρ=(1−ϕᵥ)ρₛ+φᵥρ_{g} (23)

and

c=(1−φᵥ)cₛ+ϕᵥc_{g} (24)

The thermal conductivity, however, is affected by the microstructural details and a calculation of the exact thermal conductivity is highly involved.

The analysis is limited here to the calculation of an upper bound, kᵤ, and a lower bound, kₗ, of the thermal conductivity. The upper-bound may be expressed as:

kᵤ=(1−ϕᵥ)kₛ+ϕᵥk_{g} (25)

and the lower-bound may be written as:

$$\frac{1}{{k}_{l}} = \frac{( 1 - {φ}_{v} )}{{k}_{s}} + \frac{{φ}_{v}}{{k}_{g}} \qquad ( 26 )$$

For the material properties representative of the conditions of the non-limiting experimental examples presented later (cₛ=1000 J/kgK, c_{g}=1005 J/kgK, ρₛ=1150 kg/m³, ρ_{g}=1.8×10⁻⁵ kg/m³, kₛ=0.1 W/mK, k_{g}=0.026 W/mK, T_{w}=25° C.), the temperature at the center of a plasticized foam with H₀=6 mm and φᵥ=0.5 is reduced from T₀=80° C. to T_{c}=70° C. in about 15-23 seconds.

This result may be combined with the model for the terminal rising bubble velocity introduced in the previous section. By Eq. (21), a bubble with radius R_{b}=400 μm rises by less than 300 μm in 23 seconds if the melt viscosity is greater than about 46 Pa·s. Thus to preserve the microstructure reasonably well after the foam is dispensed in the mold, the viscosity of the melt should be greater than about 46 Pa·s in this non-limiting example.

### (k) Summary of Process Models

The above non-limiting models demonstrate that the size and volume fraction of cells in the cellular solids produced by the process presented are predictable by the gas pressure, the pulsing parameters of the on/off valve, the flow rate of melt or plasticized material in the extrusion channel, and the geometry of the extrusion channel and the internally hollow needle. Thus the microstructure of the cellular solids produced by the process presented can be precisely controlled. This enables the design of cellular solids with unique, precisely controlled, and deterministic properties. The process presented can further be operated at a variety of process rates in a continuous, semi-continuous, or batch mode. Moreover, the process is highly economical and process times of the order of a minute, or even less, are achievable in some embodiments.

### Elements of the Method and Apparatus

In view of the theoretical considerations and examples above, which are suggestive and approximate rather than exact, the aspects and embodiments of the present invention may further include the following elements.

In any aspect of the method and apparatus disclosed herein, the extrusion channel cross section may be uniform or non-uniform along its length. In some embodiments, the extrusion channel cross section tapers down before the exit port to the cross section of said exit port. Furthermore, in some embodiments the extrusion channel bifurcates into at least one other end comprising an exit port. Multiple exit ports are desirable for achieving high process rates.

In some embodiments of the aspects disclosed herein, a fraction of the housing is optically transparent. Said fraction is between 0 and 1 in the invention herein. A partially or entirely optically transparent housing may enable improved optical sensing of the state and microstructure of the material (e.g., the one or more granular solids, the plasticized matrix, or the plasticized foam) in the extrusion channel. Non-limiting examples of materials which an optically transparent fraction of the housing may consist of include one of glass (e.g., silicon dioxide, borosilicate, alumina, germanium dioxide, spectrosil quartz, etc.) or plexiglass (e.g., poly(methyl methacrylate)). Non-limiting examples of materials which a non-optically-transparent fraction of the housing may consist of include one of metals, such as various forms of steel, stainless steel, iron, aluminum, tungsten, iridium, nickel, platinum, copper, or alloys or combinations thereof, among others.

In some embodiments, the apparatus or method herein comprises a granular solid feeding unit. Said granular solid feeding unit injects one or more granular granular solids into the extrusion channel. By way of example but not by way of limitation, said granular solid feeding unit may comprise a device that is capable of controlling the rate at which the one or more granular solids are injected by either volumetric or gravimetric control. In another non-limiting example, the granular solid feeding unit comprises a hopper that may be filled with one or more granular solids. The flow of the one or more granular solids from the hopper into the extrusion channel may be driven by gravity in this case. Any more examples of a granular solid feeding unit would be obvious to a person of ordinary skill in the art. All of them are within the scope of this invention.

The apparatus or method disclosed herein may comprise a plasticized matrix feeding unit for injecting a plasticized matrix into the extrusion channel. By way of example but not by way of limitation, said plasticized matrix feeding unit may comprise a device that is capable of controlling the rate at which a plasticized matrix is injected by either volumetric or gravimetric control. Any more examples of a plasticized matrix feeding unit would be obvious to a person of ordinary skill in the art. All of them are within the scope of this invention.

In some embodiments of the apparatus or method of this invention, the housing further comprises at least one other feeding port for injecting cell-fluid into the extrusion channel. Multiple cell-fluid feeding ports are desirable for achieving high process rates.

Also, in some embodiments the apparatus comprises at least one heating element. The heating element can be a wrap around heater in some cases. A non-limiting example of such a wrap around heater is a band heater wrapped around the housing. In some embodiments, the heating element may be embedded into the housing, such as one or more cartridge heaters fixed in and surrounded by the housing, among others. The heating element may also be partially embedded into the housing, such as fluid channels in the housing filled with a circulating fluid, said circulating fluid being temperature-controlled by an external temperature control unit. Such a system may, for example, not only permit heating, it may also allow to cool the housing if necessary and thereby enable improved temperature control. It may be noted that in the context of this disclosure, all such heating elements that are “partially embedded into the housing” are considered “embedded into the housing”. Thus in some embodiments of the method disclosed herein, the heating may be performed using a wrap around heater, or a heater embedded into the housing, or a furnace, or any combinations thereof, among others. Any more examples of a heating elements would be obvious to a person of ordinary skill in the art. All of them are within the scope of this invention.

In some embodiments, the apparatus disclosed herein comprises a solvent feeding unit attached to a feeding port for injecting at least one solvent into the extrusion channel. By way of example but not by way of limitation, a solvent feeding unit may comprise any device to move/dispense/inject solvent at a controlled rate into the extrusion channel, such as a peristaltic pump, a diaphragm pump, a rotary vane pump, a syringe pump, or any other rotary or positive displacement pump, among others. Any more examples of a solvent feeding unit would be obvious to a person of ordinary skill in the art. All of them are within the scope of this invention.

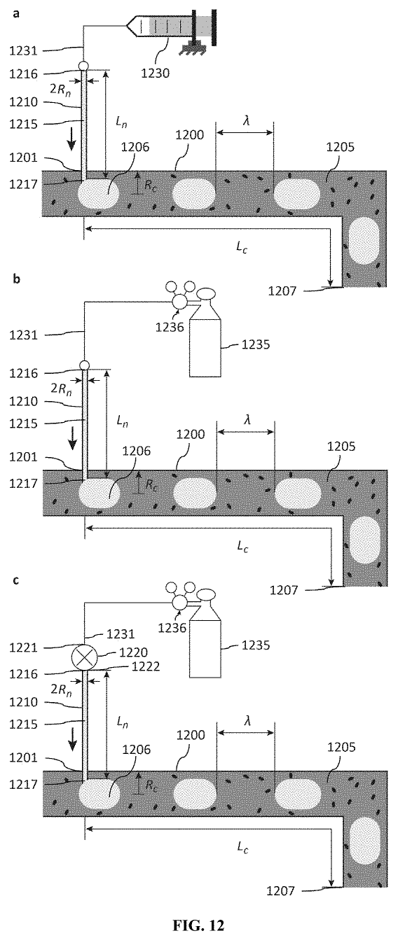

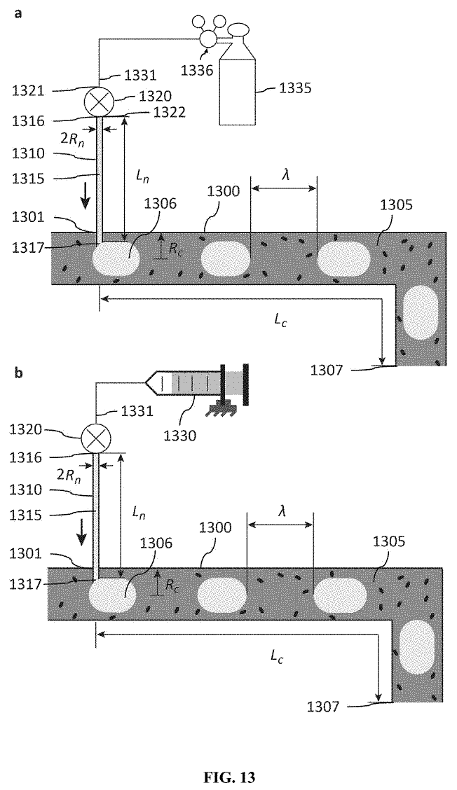

FIGS. 12 and 13 present non-limiting examples of cell-fluid feeding units according to this invention. A cell-fluid feeding unit may comprise at least one internally hollow needle 1210, 1310 having an internal surface encapsulating and defining a microfluidic channel 1215, 1315 having a first needle channel end 1216, 1316 and a second needle channel end 1217, 1317 and a cross section extending axially along its length from said first needle channel end 1216, 1316 to said second needle channel end 1217, 1317 and terminating into a needle channel exit port at the second needle channel end 1217, 1317. Thus the cell-fluid injection into the plasticized matrix in the extrusion channel 1205, 1305 may be performed using at least one internally hollow needle 1210, 1310 as just described. It may be noted that the wall thickness of the internally hollow needle 1210, 1310 may be uniform or non-uniform along the length of the microfluidic channel 1215, 1315. Also, the wall thickness of the needle may range from about a micrometer or less to several meters. In such cases where the wall thickness of the internally hollow needle is much greater than the hydraulic diameter of the microfluidic channel 1215, 1315, the internally hollow needle 1210, 1310 may also be considered a solid block, rod, slab, bar, piece, etc. with an internal microfluidic channel 1215, 1315. Thus, the outer shape of an internally hollow needle may assume any shape and geometry. It may be noted, however, that if a fraction of the internally hollow needle is surrounded by the extrusion channel, said fraction may have an outer shape so that the flow of plasticized matrix or plasticized foam along the extrusion channel is not impaired strongly or excessively.

The second needle channel end 1217, 1317 of said microfluidic channel 1215, 1315 may be embedded in or attached to the housing 1200, 1300 or a feeding port 1201, 1301 for injecting cell-fluid into the extrusion channel 1205, 1305. Furthermore, in some embodiments, said microfluidic channel 1215, 1315 bifurcates into at least one other needle channel end comprising a needle channel exit port 1217, 1317. Multiple needle channel exit ports for injecting cell-fluid into the extrusion channel 1205, 1305 are desirable for achieving high process rates. Also, in any aspect of the invention herein, the cross section of said microfluidic channel 1215, 1315 may be uniform or non-uniform along its length.

In other words, in some embodiments the the cell-fluid injection unit comprises a cell-fluid injection channel 1215, 1315 having a first end 1216, 1316 and at least a second end 1217, 1218 contiguous with and terminating in the extrusion channel 1205, 1305. Furthermore, in some embodiments the cell-fluid injection is performed using a cell-fluid injection channel 1215, 1315 having a first end 1216, 1316 and at least a second end 1217, 1317 contiguous with and terminating in the extrusion channel 1205, 1305.

The area of the cross section of the microfluidic channel 1215, 1315 at a needle channel exit port 1217, 1317 (e.g., the area of the cross section of the cell-fluid injection channel 1215, 1315 at one or more of its second ends 1217, 1317) is a factor that determines the lower-limit of the volume of the injected cell 1206, 1306 (e.g., a bubble or a drop). A small cross sectional area at the second end of the cell-fluid injection channel 1217, 1317 is therefore desirable for injecting small cells 1206, 1306 into the plasticized matrix or foam in the extrusion channel 1205, 1305. The cross sectional area of the second end of the cell-fluid injection channel 1217, 1317 should, however, be large enough to avoid clogging of the second end of the cell-fluid injection channel 1217, 1317 during operation. Thus, in some embodiments herein, the cross sectional area of the cell-fluid injection channel (e.g., a microfluidic channel) 1215, 1315 may increase before a second end of the cell-fluid injection channel 1217, 1317 to the cross sectional area of said second end 1217, 1317.

In some embodiments, as shown schematically in FIG. 12a, the cell-fluid injection unit further comprises at least one cell-fluid pump 1230 to perform the cell-fluid injection. The cell-fluid pump 1230 may comprise any device to move/dispense/inject cell-fluid at a controlled rate, such as a peristaltic pump, a diaphragm pump, a rotary vane pump, a syringe pump, or any other rotary or positive displacement pump, among others.

In some embodiments, the cell-fluid pump 1230 may have at least one cell-fluid supply port 1231 attached to or connected to a first end of the cell-fluid injection channel 1216 so as to inject cell-fluid through the cell-fluid injection channel 1215 into a plasticized matrix or foam in the extrusion channel 1205. The cell-fluid pump may inject cell-fluid through the cell-fluid injection channel 1215 into a plasticized matrix or foam in the extrusion channel 1205 by generating a cell-fluid pressure in the supply port 1231 or in the cell-fluid injection channel 1215. By way of example but not by way of limitation, said cell-fluid pressure may be constant (or essentially constant), or it may be pulsatile. A pulsatile pressure in the supply port 1231 or cell-fluid injection channel 1215 enables to control the size or volume of the injected cells 1206 and the repetition rate of cell injection independently (e.g., distinctively) and precisely. Thus, in some embodiments the cell-fluid injection unit further comprises a device for the application of pressure pulses in the cell-fluid injection channel 1215 so as to control the volume of injected cells and the repetition rate of cell injection. In the invention herein, the pressure of cell-fluid is usually understood “pulsatile” (e.g., the pressure of cell-fluid in the cell-fluid injection channel 1215, or the pressure of cell-fluid in the supply port 1231, etc. is pulsatile) if the cell-fluid is primarily injected into the plasticized matrix in the extrusion channel 1205 during one or more pressure pulses (e.g., durations of elevated pressure in the cell-fluid injection channel 1215, or durations of elevated pressure in the supply port 1231, etc.). In between pressure pulses, however, no, or essentially no, or less cell fluid may be injected.

In some embodiments of the apparatus and method disclosed herein, as shown schematically in the non-limiting FIG. 12b, the cell-fluid injection unit comprises at least one internally hollow cell-fluid reservoir 1235 to perform the cell-fluid injection. Said at least one cell-fluid reservoir 1235 may be filled with cell-fluid and have at least one cell-fluid supply port 1231 attached to at least one first end of a cell-fluid injection channel 1216.

The rate at which cell-fluid is injected into the extrusion channel 1205 may be controlled by the pressure of said cell-fluid in said at least one first end of a cell-fluid injection channel 1216 or the pressure of cell-fluid in said at least one cell-fluid supply port 1231. By way of example but not by way of limitation, said pressures may be controlled by a pressure regulator 1236 connected to or attached to said at least one cell-fluid reservoir 1235. The rate at which cell-fluid is injected into the extrusion channel 1205 may further be controlled by the flow resistance of the cell-fluid injection channel 1215 and additional flow resistors or actuators, among others.

In any case, during cell-fluid injection into the extrusion channel 1205, the cell-fluid pressure in said at least one cell-fluid supply port 1231 (and the cell-fluid pressure in the cell-fluid injection channel 1215, 1216 and in the reservoir 1235) may be greater (e.g., at least 1.05 times greater, or at least 1.1 times greater, or at least 1.2 times greater, or at least 1.3 times greater, or at least 1.4 times greater) than the pressure of the plasticized matrix or foam in the extrusion channel 1205 at a feeding port 1201 for injecting cell-fluid.

Furthermore, in some embodiments the cross section along the length of the cell-fluid injection channel 1215 may be so that the flow resistance of said cell-fluid injection channel 1215 is greater than 1×10⁹ Pa·s/m³. This includes, but is not limited to a flow resistance greater than 5×10⁹ Pa·s/m³, or greater than 1×10¹⁰ Pa·s/m³, or greater than 5×10¹¹ Pa·s/m³, or greater than 7.5×10¹⁰ Pa·s/m³, or greater than 1×10″ Pa·s/m³, or greater than 5×10¹¹ Pa·s/m³, or greater than 7.5×10¹¹ Pa·s/m³, or greater than 1×10¹² Pa·s/m³.

In the context of this disclosure, the flow resistance of a microfluidic channel 1215 is determined by allowing water at a temperature of 25° C. to flow from the first needle channel end 1216 to a needle channel exit port 1217. The flow rate of water, Q_{w}, through the needle channel exit port 1217 and the pressures at the first needle channel end 1216, P_{f}, and at the needle channel exit port 1217, Pₑ, may be measured. The resistance, Res, can then be determined as Res=(P_{f}−Pₑ)/Q_{w}.

Moreover, as shown schematically in FIG. 12c, for improved control of the volume of the injected cells 1206 and the rate at which cells 1206 are injected, in some embodiments the cell-fluid injection unit further comprises at least one flow resistor 1220. By way of example but not by way of limitation, the first end of the cell-fluid injection channel 1216 may be connected to or attached to said at least one flow resistor 1220.

In some embodiments, said at least one flow resistor 1220 comprises a constant (e.g., a time-invariant) flow resistance.

Moreover, in some embodiments the flow resistance of said at least one flow resistor 1220 divided by the number of second ends of the cell-fluid injection channel 1217 (e.g., the number of second ends 1217 of the cell-fluid injection channel 1215 said flow resistor 1220 is connected to) may be greater than 1×10⁹ Pa·s/m³. This includes, but is not limited to a flow resistance divided by the number of second ends 1217 of the cell-fluid injection channel 1215 greater than 5×10⁹ Pa·s/m³, or greater than 1×10¹⁰ Pa·s/m³, or greater than 5×10″ Pa·s/m³, or greater than 7.5×10¹⁰ Pa·s/m³, or greater than 1×10″ Pa·s/m³, or greater than 5×10″ Pa·s/m³, or greater than 7.5×10¹¹ Pa·s/m³, or greater than 1×10¹² Pa·s/m³. In the context of this disclosure, the flow resistance of said at least one flow resistor 1220 is determined by allowing water at a temperature of 25° C. to flow from the first end of the resistor 1221 to the second end of the resistor 1222. The flow rate of water, Q_{wR}, and the pressures at the first end 1221, P_{fR}, and at the second end 1222, P_{sR}, of the resistor 1220 may be measured. The resistance, Res_{R}, of the resistor 1220 can then be determined as Res_{R}=(P_{fR}−P_{sR})/Q_{wR}.

It may be obvious to a person of ordinary skill in the art that multiple flow resistors may be connected in series. Thus, in some embodiments of the invention herein, all the serial flow resistors in their totality, (e.g., the sum of all the flow resistors that are connected in series), have a flow resistance divided by the number of second ends of the cell-fluid injection channel 1217 greater than 1×10⁹ Pa·s/m³. This includes, but is not limited to a flow resistance divided by the number of second ends of the cell-fluid injection channel 1217 greater than 5×10⁹ Pa·s/m³, or greater than 1×10¹⁰ Pa·s/m³, or greater than 5×10¹⁰ Pa·s/m³, or greater than 7.5×10¹⁰ Pa·s/m³, or greater than 1×10¹¹ Pa·s/m³, or greater than 5×10¹¹ Pa·s/m³, or greater than 7.5×10¹¹ Pa·s/m³, or greater than 1×10¹² Pa·s/m³.

For achieving even better control of the cell 1306 volume and the rate at which cells 1306 are injected into the plasticized matrix or foam, in some embodiments at least one flow resistor 1320 comprises a time-variant flow resistance (FIG. 13a). Said flow resistor 1320 with time-variant flow resistance may, for example, comprise an on/off valve, also referred to herein as “pulse valve”. The on/off valve may have at least an on-state (e.g., an open state) and at least an off-state (e.g., a closed state), said on-state permitting cell-fluid injection from a pressure reservoir 1331, 1335 into the plasticized matrix in the extrusion channel 1305, said off-state restricting cell-fluid injection from said pressure reservoir 1331, 1335 into the plasticized matrix in the extrusion channel 1305.

It may be noted that during the on-state of the valve 1320 the pressure in the cell-fluid injection channel 1315 may be greater than the pressure of the plasticized matrix in the extrusion channel 1205 at the location of cell injection (also referred to herein as “feeding port 1201 for injecting cell-fluid”). During the off-state of the valve 1320, however, the pressure of cell-fluid in the cell-fluid injection channel 1315 may be about the same as the pressure of the plasticized matrix in the extrusion channel 1305 at the location of cell injection 1301. Thus a resistor 1320 with time-variant flow resistance (e.g., an on/off valve or a pulse valve) connected to a pressure reservoir 1331, 1335 is a non-limiting example of a device for the application of pressure pulses in the cell-fluid injection channel 1315 so as to control the volume of injected cells 1306 and the repetition rate of cell injection.

Moreover, in some embodiments, the state of an on/off valve (e.g., a pulse valve or a device for the application of pressure pulses in the cell-fluid injection channel) is repeatedly switched between said on-state and said off-state during the operation of at least one conveying element. The opening time of the valve (e.g., the time of the on-state) in one repetition period may be no greater than 5 seconds. This includes, but is not limited to an opening time in one repetition period no greater than 4 seconds, or no greater than 3 seconds, or no greater than 2 seconds, or no greater than 1 second, or no greater than 0.8 seconds, or no greater than 0.6 seconds. The closing time of the valve (e.g., the time of the off-state) in one repetition period may be no greater than 20 seconds in some embodiments. This includes, but is not limited to a closing time in one repetition period no greater than 15 seconds, or no greater than 10 seconds, or no greater than 5 seconds, or no greater than 2 seconds, or no greater than 1 second, or no greater than 0.5 seconds. In some embodiments, both the opening and closing times of the valve are at the micro-to-milli-second scale. Thus the number of cells 1306 injected per second (e.g., the repetition rate at which cells are injected) is of the order of about 100-100,000 l/s in these non-limiting embodiments.

If the injected cell-fluid is compressible, and the first end of the cell-fluid injection channel 1316 connected to an on/off valve, the volume of the cell-fluid injection channel 1315 should generally be small for achieving precise control of the volume and rate at which cells (e.g., bubbles or drops) are injected. Thus, in some embodiments the volume of a cell-fluid injection channel 1315 divided by the number of its second ends 1317 contiguous with and terminating in the extrusion channel 1305 is no greater than 200 mm³. This includes, but is not limited to a volume of said cell-fluid injection channel 1315 divided by the number of its second ends 1317 contiguous with and terminating in the extrusion channel 1305 no greater than 125 mm³, or no greater than 85 mm³, or no greater than 50 mm³, or no greater than 25 mm³, or no greater than 10 mm³, or no greater than 5 mm³, or no greater than 2 mm³, or no greater than 1 mm³, or no greater than 0.5 mm³. It may be noted further that in some non-limiting cases it is desirable that the volume of the cell-fluid injection channel 1315 divided by the number of its second ends 1317 contiguous with and terminating in the extrusion channel 1305 is no greater than the desired volume of the injected cell 1306.

A resistor 1320 with time-variant flow resistance may also be connected in series with a cell-fluid pump 1330 and a cell-fluid injection channel 1315 as shown in FIG. 13b. The time-variant flow resistor 1320 may provide improved control for injection of cells 1306 with a specific volume at discrete times. Generally, cell-fluid injection units with a pulse valve or time-variant flow resistor enable a greater range and better control of injected cell volume and repetition rate than systems without.

It may be noted that in some embodiments, the internally hollow needle 1310 may be connected to an actuator to disturb the fluid stream in the cell-fluid injection channel 1315 so as to promote break-up of the cell-fluid stream injected into the plasticized matrix or foam. By way of example but not by way of limitation, such an actuator may be a piezo-electric actuator. In the context of this disclosure, all such elements to promote break-up of the cell-fluid stream (e.g., actuators, time-variant flow resistors, etc.) are referred to as “device for controlling the volume and repetition rate of the injected cells” or “device for the application of pressure pulses in the cell-fluid injection channel so as to control the volume of injected cells and the repetition rate of cell injection”. Any more examples of such devices for the application of pressure pulses in the cell-fluid injection channel so as to control the volume of injected cells and the repetition rate of cell injection would be obvious to a person of ordinary skill in the art. All of them are within the scope of this invention.

In some embodiments of the method or apparatus disclosed herein, the volume of an injected cell 1206, 1306 in the plasticized foam is no greater than 200 mm³. This includes, but is not limited to a volume of an injected cell 1206, 1306 in the plasticized foam no greater than 125 mm³, or no greater than 85 mm³, or no greater than 50 mm³, or no greater than 25 mm³, or no greater than 10 mm³, or no greater than 5 mm³, or no greater than 2 mm³, or no greater than 1 mm³.

Also, in some embodiments, cell-fluid is injected into the plasticized matrix or the plasticized foam during the operation of at least one conveying element. This ensures that the injected cells 1206, 1306 are transported away from the feeding port 1201, 1301 for injecting cell-fluid so that a new, discrete cell 1206, 1306 can be injected into the plasticized material. Thus, in some embodiments the mean free distance, A, between an injected cell 1206, 1306 and its neighboring cell in the plasticized foam is no greater than 100 mm. This includes, but is not limited to a mean free distance, A, between an injected cell 1206, 1306 and its neighboring cell in the plasticized foam no greater than 50 mm, or no greater than 20 mm, or no greater than 10 mm, or no greater than 5 mm, or no greater than 2 mm, or no greater than 1 mm.

Furthermore, as the plasticized foam in the extrusion channel 1205, 1305 flows towards an exit port 1207, 1307, in some embodiments the average velocity of the plasticized foam in the extrusion channel 1205, 1305 with respect to the housing 1200, 1300 is greater than 0.1 mm/s. This includes, but is not limited to an average velocity of the plasticized foam in the extrusion channel 1205, 1305 with respect to the housing 1200, 1300 greater than 0.25 mm/s, or greater than 0.5 mm/s, or greater than 1 mm/s, or greater than 2 mm/s, or greater than 5 mm/s. For a given cross section of the extrusion channel 1205, 1305, the greater the velocity of the plasticized foam is in the extrusion channel 1205, 1305, the greater is the rate at which the cellular solids are manufactured.

In some embodiments of the apparatus disclosed herein, the conveying element is a fluid pump (e.g., a device that moves fluids or granular solids or a plasticized matrix), such as a peristaltic pump, a diaphragm pump, a rotary vane pump, an extrusion gear pump, etc. Other non-limiting examples of the conveying element include a piston, a screw, or pressurized gas. Thus in the method herein, mechanical work may be applied on the plasticized matrix using a fluid pump, a piston, a screw, or pressurized gas, among others. Furthermore, in any aspect of the apparatus or method of this invention, the fluid pump, piston, or screw may be operated mechanically (e.g., hydraulically or pneumatically) or with a motor, such as an electrical AC motor, DC motor, or stepper motor, among others.

In some embodiments, at least one conveying element extrudes the plasticized foam through an exit port for dispensing into a mold. Also, in some non-limiting embodiments the plasticized foam solidifies in the mold and then the cellular solid may be ejected from the mold. By way of example but not by way of limitation, solidification of the plasticized foam may be by cooling the plasticized foam to below its melting temperature, by removing solvent from the plasticized foam, or by cross-linking some of the constituents of the plasticized foam.

Non-limiting examples of the mold include an open cavity, a closed cavity, a curved solid surface, or even a flat solid surface. The mold or surface may be movable. Also, in some embodiments the mold or surface may be temperature-controlled.

In some embodiments, the one or more granular solids are selected from the group of pharmaceutical excipients, active pharmaceutical ingredients, foods, sweeteners, proteins, polymers, metals, or ceramics.

In some embodiments, the shear viscosity of the plasticized matrix in the extrusion channel is greater than 0.01 Pa·s. This includes, but is not limited to a shear viscosity of the plasticized matrix in the extrusion channel greater than 0.025 Pa·s, or greater than 0.05 Pa·s, or greater than 0.075 Pa·s, or greater than 0.1 Pa·s, or greater than 0.5 Pa·s, or greater than 1 Pa·s, or greater than 2 Pa·s, or greater than 5 Pa·s, or greater than 10 Pa·s.

In some embodiments, the cell-fluid is a gas. This includes, but is not limited to one of air, oxygen, nitrogen, CO₂, or argon. The cell-fluid may also be a liquid in some non-limiting embodiments herein. By way of example but not by way of limitation, this includes water, milk, olive oil, or sesame oil, among others. In some embodiments, the cell-fluid may solidify either in the plasticized foam or in the cellular solid after injection into the plasticized matrix or foam. In this case, the cells are solid particulates surrounded by the walls and edges of the plasticized foam or the cellular solid.

In some embodiments of the invention herein, the cellular solid comprises one or more solid constituents, said solid constituents comprising a three-dimensional network (e.g., a three dimensional structure or framework or skeleton) of walls and edges, said walls and edges defining one or more cells in said three dimensional network of walls and edges; wherein the average cell size is 0.1 μm-10 mm; the average wall or edge thickness is 0.1 μm-10 mm; and the volume fraction of cells with respect to a representative control volume of the cellular solid is 0.05-0.95. This includes, but is not limited to an average cell size of 0.5 μm-7 mm, or 0.5 μm-5 mm, or 0.5 μm-3 mm, or 1 μm-5 mm, or 0.75 μm-3 mm, or 1 μm-3 mm and an average wall or edge thickness of 0.5 μm-7 mm, or 0.5 μm-5 mm, or 0.5 μm-3 mm, or 1 μm-5 mm, or 0.75 μm-3 mm, or 1 μm-3 mm and a volume fraction of cells with respect to a representative control volume of the cellular solid of 0.05-0.9, or 0.1-0.9, or 0.1-0.95, or 0.15-0.95, or 0.15-0.9, or 0.15-0.85, or 0.2-0.85.

In some embodiments, the cellular solid comprises a pharmaceutical dosage form (e.g., a tablet or a capsule for oral ingestion, etc.).

### EXPERIMENTAL EXAMPLES

The following examples present ways by which the cellular solids and the method and apparatus for manufacture thereof may be prepared, performed, and analyzed, and will enable one of skill in the art to more readily understand the invention. The examples are presented by way of illustration and are not meant to be limiting in any way.

### Example 1: Apparatus for Preparing Non-Porous and Cellular Solids

Non-limiting experimental examples of non-porous (or minimally-porous) and cellular solids were prepared by an adapted desktop screw extruder with the following components: a granular solid feeding unit, an extrusion screw and barrel, a gas delivery unit, and a molding unit. The granular solid feeding unit consists of a syringe with uniform barrel diameter and a syringe pump to deliver the granules into a hopper. The volumetric flow rate is determined by the velocity of the syringe's piston. The hopper directs the granules into the extrusion screw and barrel. The extrusion screw is 244 mm long, has an outer diameter of 10 mm, a helix angle of 17.65°, a channel height of 1 mm, and a channel width of 8.5 mm. The barrel is surrounded by a resistance heater coil to set the temperature of the barrel. The material exiting the extruder is delivered into a nozzle with 1 mm diameter. The nozzle is made of Plexiglas (to facilitate imaging) and heated by an infrared lamp. A hypodermic needle with an inner diameter of 50 μm and 25 mm long is inserted into the channel. The needle is connected to a pulse valve (on/off valve) in line with a gas regulator and cylinder. The material exiting the nozzle flows downward into a cylindrical mold. FIG. 14 is a photograph of the apparatus.

### Example 2: Preparation of Non-Porous and Cellular Solids

The non-porous and cellular solids were prepared by first mixing 5 wt % of solid Acetaminophen particles (a drug) with 95 wt % granules of PEG 35,000 or PEG 35k (an excipient). The particles were mixed and loaded in the granule-feeding unit (a syringe in a syringe pump) which was set do deliver 1.73 mg/s. The rotation rate of the screw was about 3 rpm and the barrel and nozzle temperatures were set to 80° C. The temperature of the ambient air and the mold were 25° C.

For preparing the non-porous (or minimally-porous) solids, the pulse valve was closed. The mold was filled with the effluent stream until a height of 6 mm was reached in the mold. The material was then left in the mold for about two minutes to solidify. Subsequently, the final dosage form was ejected from the mold. For preparing the cellular solids, the pulse valve was operated at various combinations of τ_{g} and τₘ. The range of τ_{g} was 20-60 ms and that of τₘ 400-600 ms. Again, the mold was filled with the effluent stream until a height of 6 mm was reached. The material was then left in the mold for about 2 minutes to solidify and afterwards, the cellular solid was removed from the mold. All the solids prepared in this work were cylindrical disks with the same dimensions: 9 mm in diameter and about 6 mm in thickness.

### Example 3: Images of Process Steps

The process steps that were imaged include gas injection in the nozzle, transport of gas bubbles to the nozzle exit, fluid stream effluent from the nozzle, and deposition and build-up of the foam on a horizontal surface (the mold was removed for imaging). Images were taken by a Nikon DX camera.

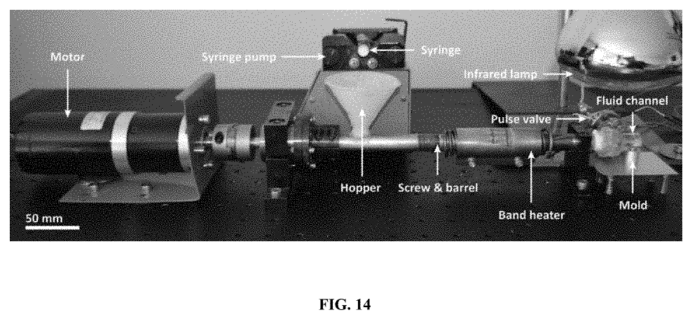

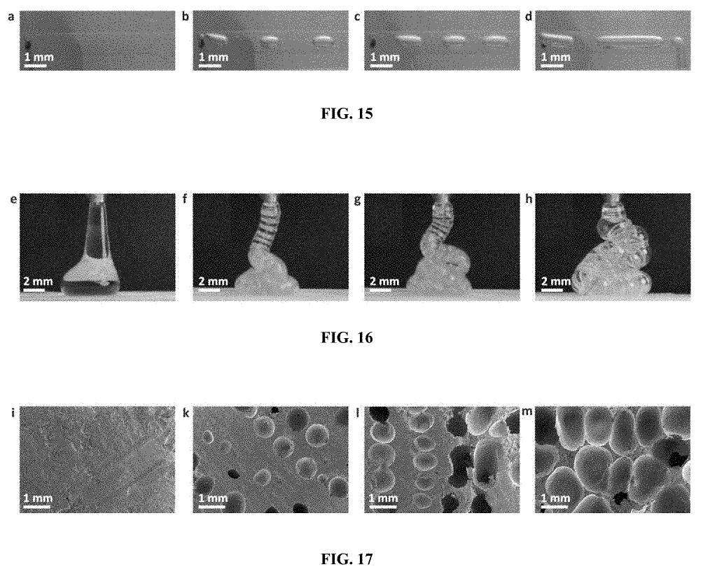

Images of the melt stream after gas injection are presented in FIGS. 15a-8d for different pulsing parameters of the gas flow. FIG. 15a shows the melt stream in the channel without any injected gas. The stream is bubble-free.

A representative image of the melt stream with gas pulses of τ_{g}=20 ms and τₘ=600 ms (condition B) is shown in FIG. 15b. The average bubble volume, V_{b}=0.15 mm³ and the distance between the bubbles λ=1.397 mm (Table 1). FIGS. 15c and 15d demonstrate that V_{b} increases if τ_{g} is increased, and λ decreases if τₘ is decreased. For example, if τ_{g}=60 ms and τₘ=400 ms, V_{b}=0.766 mm³ and λ=676 μm (condition D, FIG. 15d and Table 1).

These results are compared with the values calculated based on the process parameters and the process models presented above. The bubble volume right at the exit of the needle is calculated by Eq. (16) (Table 1). The calculated and measured values agree if a 40% pressure drop (i.e., a 40% volumetric expansion) of the bubble is assumed between the needle exit and the location of the bubble in the channel shown in FIGS. 15b-15d.

The “free” distance between the bubbles, λ, is calculated by Eq. (13). As shown in Table 1, the calculated values deviate by less than 25 percent from the experimental values.

The fluid stream exiting the channel was allowed to accumulate on a horizontal surface (with the mold removed) as shown in FIGS. 16e-16h. Without any added gas, there were no bubbles in the fluid thread. A clear liquid was deposited on the bottom surface as shown in FIG. 16e.

When gas was added upstream, the bubbles were visible and assumed the shape of a disk, or cylinder, in the fluid thread (FIGS. 16f-16h). The foam deposited on the flat surface first grew vertically and then radially before it solidified.

### Example 4: Microstructures of Cellular Solids

A cross-sectional SEM image of the dosage form was obtained by cutting it with a thin microtome blade (MX35 Ultra, Thermo Scientific, Waltham, Mass.). A Zeiss Merlin High Resolution SEM with a GEMINI column was then used for imaging the microstructure. Imaging was done with an in-lens secondary electron detector. An accelerating voltage of 5 kV and a probe current of 95 pA were applied to operate the microscope.

Scanning electron microscopy images of the solidified material structure are presented in FIGS. 17i-17m. The cast solid dosage forms (condition A) are essentially non-porous as shown in FIG. 17i. The drug and excipient cannot be distinguished in the image.

For an analysis of the microstructural details of the cellular solids, a number of lines were drawn randomly on each SEM image of the microstructure (FIGS. 17i-17m). The mean linear intercept, I_{cell}, which is the ratio of the total length covered by the cells, L_{cell}, and the number of cells that intersect with the lines, N_{cell}, was then determined for each microstructure:

$${l}_{cell} = \frac{{L}_{cell}}{{N}_{cell}} \qquad ( 27 )$$

Assuming that the cells are spherical and of uniform size,

$${l}_{cell} = 4 \frac{{V}_{cell}}{{S}_{cell}} = \frac{2}{3} {D}_{cell} \qquad ( 28 )$$

where V_{cell} is the volume of a single cell, S_{cell} its surface area, and D_{cell} the cell diameter.

The results of the cell diameter obtained from FIGS. 17i-17m and Eqs. (27) and (28) are listed in Table 1. D_{cell}=824 μm for the cellular solid prepared under condition B, and D_{cell}=1438 μm for condition D. The cell size calculated by combining Eqs. (16)-(18) is 833 μm for condition B and 1664 μm for condition D (Table 1). Thus the calculated values agree with the experimental results fairly well.

The volume fraction of voids in the solidified cellular solids, coy, was determined from their density, ρ, as:

$${φ}_{v} = 1 - \frac{ρ}{{ρ}_{s}} \qquad ( 29 )$$

where ρₛ is the density of the non-porous solid. The densities of the cellular solids were determined by measuring their weight and volume. The volume fraction of voids of each cellular solid was measured in triplicate. As tabulated in Table 1, the measured φᵥ=0.26 for the cellular solid B. The measured φᵥ increases to 0.72 for the cellular solid D.

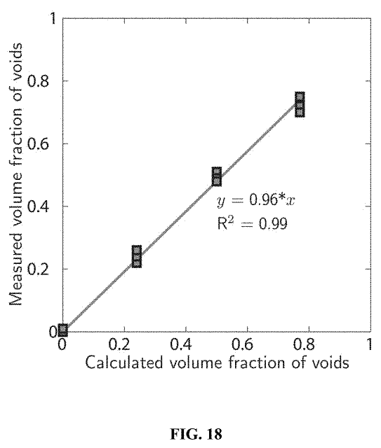

A plot of the measured versus calculated volume fraction of voids is shown in FIG. 18 (φᵥ is calculated by Eq. (19) and Eqs. (8), (10), (11), and (15) using the parameter values given in Table 1). The data can be fitted to a curve y=0.96*x with an R² value of 0.99. This confirms the agreement between calculations and experimental values.

The mean free distance between the cells, λ_{cell}, may be written as:

$${λ}_{cell} = \frac{1 - {φ}_{v}}{{N}_{cell} / ( {L}_{cell} + {L}_{solid} )} = \frac{( {L}_{cell} + {L}_{solid} ) ( 1 - {φ}_{v} )}{{N}_{cell}} \qquad ( 30 )$$

The values obtained for λ_{cell} range from 271 μm for the cellular solid D to 1.5 mm for the cellular solid B (Table 1).

Finally, the fraction of cells that are open, fₒₚₑₙ, was determined by counting the number of cells with an open passage to another cell on the SEM image, and dividing this number with the total number of cells. The value for fₒₚₑₙ so obtained is approximate, because the SEM images do not show the three-dimensional view of the cells. The estimated fₒₚₑₙ is between less than 0.2 for the cellular solid B and 0.55 for the cellular solid D.

### Example 5: Drug Release

In the context of this non-limiting example, the non-porous and cellular solids are used as pharmaceutical dosage forms or just “dosage forms”. Thus the terms “cellular solid” and “cellular dosage form” are used interchangeably. Also, the terms “non-porous solid” and “non-porous dosage form” or “non-porous solid dosage form” are used interchangeably.

Drug release of the non-porous and cellular dosage forms was tested by first placing the dosage form in a basket in a dissolution vessel (a USP dissolution apparatus 1 from Sotax AG) filled with 900 ml of 0.05 M phosphate buffer solution (using sodium phosphate monobasic and sodium phosphate dibasic) at a pH of 5.8 and at 37° C. The basket was rotated at 50 rpm. The concentration of dissolved drug was measured versus time by UV absorption at 244 nm using a fiber optic probe (Pion, Inc.). Drug release experiments were done in triplicate.

TABLE 1
Summary of process parameters, and calculated and measured process and property values for experimental/theoretical
examples given.

| | Process | | | Calculated process and | | | | | | Measured | | | | | | |
|---|---|---|---|---|---|---|---|---|---|---|---|---|---|---|---|---|
| | parameters | | | microstructural parameters | | | | | | microstructural parameters | | | | | | |
| | P_{g} | τ_{g} | τₘ | P_{m,F} | λ | V_{b,F}* | D_{eq}* | D_{cell} | | λ | V_{b}* | D_{eq}* | D_{cell} | | λ_{cell} | |
| | (MPa) | (ms) | (ms) | (MPa) | (μm) | (mm³) | (μm) | (μm) | φᵥ | (mm) | (mm³) | (μm) | (μm) | φᵥ | (μm) | fₒₚₑₙ |
| A | — | — | — | 0.206 | — | — | — | — | 0.00 | — | — | — | — | 0.01 | — | — |
| B | 0.35 | 20 | 600 | 0.191 | 1184 | 0.085 | 551 | 833 | 0.24 | 1397 | 0.150 | 659 | 824 | 0.26 | 1500 | <0.2 |
| C | 0.35 | 40 | 500 | 0.170 | 1031 | 0.233 | 769 | 1163 | 0.50 | 829 | 0.266 | 798 | 1002 | 0.51 | 748 | 0.55 |
| D | 0.35 | 60 | 400 | 0.139 | 879 | 0.681 | 1100 | 1664 | 0.77 | 676 | 0.766 | 1135 | 1438 | 0.72 | 271 | 0.55 |

The calculated values are derived by Eqs. (6)-(20), using the process parameters above: dM_{f}/dt = 1.73 mg/s, ρₘ = 1150 kg/m³, Q_{m,0} = 1.5 mm³/s, μₘ = 115 Pa · s, R_{c} = 0.5 mm, L_{c} = 15 mm, Rₙ = 25 μm, Lₙ = 25 mm, and Pₐₜₘ = 101 kPa. D_{eq} is the equivalent diameter of a bubble and is calculated by D_{eq} = (6V_{b,F}/π)^{0.33}.
All the measured microstructural parameters are derived from the images presented in FIG. 8, except φᵥ which is derived by Eq. (29). It may be noted that φᵥ could also be derived from the SEM images as: φᵥ = L_{cell}/(L_{cell} + L_{solid}) where L_{solid} is the total length covered by the solid along the test line(s). The results so obtained agree with the tabulated results.
*The bubble volume is calculated at the location of gas injection, but the measured bubble volume is an average of the bubble sizes downstream in the channel.

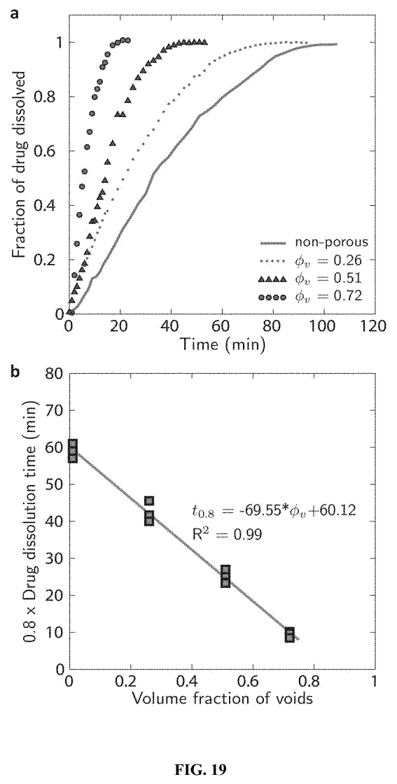

Representative results of the fraction of drug dissolved versus time after immersion of the dosage forms in the dissolution medium are shown in FIG. 19a. For all the dosage forms, the fraction of drug dissolved increased steadily with time until the entire amount of drug was dissolved, and the curves plateaud out to the final value of 1. The time required to dissolve 80% of the drug content, t_{0.8}, is extracted from these curves and plotted in FIG. 19b. t_{0.8} is about 60 minutes for the non-porous solid dosage form. It decreases with increasing volume fraction of voids and is about 10 minutes for the dosage forms with φᵥ=0.72 (condition D).

The cellular dosage forms were floating at the top of the basket during dosage form disintegration while the non-porous solid dosage forms settled at its bottom. Dosage form disintegration occurred predominantly by gradual surface erosion of the excipient with occasional removal of visible fragments. Thus, assuming that the streamlines enter the surface dimples due to the cells, the flux of the eroding polymeric excipient may be approximated by a convective mass transfer model that takes the increased surface area due to the cell dimples into account:

$$j = 0.62 \times {D}^{\frac{2}{3}} {{c}_{0} ( \frac{{ρ}_{f}}{{μ}_{f}} )}^{\frac{1}{6}} {Ω}^{\frac{1}{2}} ( 1 + {φ}_{v} ) \qquad ( 31 )$$

where D is the diffusivity of the eroding excipient polymer in the dissolution medium, c₀ the interfacial concentration, ρ_{f} and μ_{f}, respectively, are the density and viscosity of the dissolution medium, and Ω the angular velocity of the basket. If it is further assumed that erosion occurs from both faces of the dosage form, the erosion or disintegration time of the dosage form is about:

$${t}_{dis} = \frac{1}{2 } \frac{{ρ}_{s} ( 1 - {φ}_{v} ) {H}_{0}}{j} \qquad ( 32 )$$

where H₀ is the thickness of the dosage form initially.

For the dissolution medium, stirring rate, excipient, and dosage form geometry of the non-limiting example considered (c₀=271 kg/m³, D=1.09×10⁻¹⁰ m²/s, H₀=6 mm, ρ_{f}=1000 kg/m³, ρₛ=1150 kg/m³, ρ_{f}=0.001 Pa·s, and Ω=5.24 rad/s) it is estimated that 0.8×t_{dis}=52 min for the essentially non-porous dosage form, and 0.8×t_{dis}=8.4 min for the cellular dosage form with φᵥ=0.72. The calculated values of t_{dis} are fairly close to the measured data of t_{0.8} presented in FIG. 10b. It may be noted that in the non-limiting examples presented, the disintegration rates of the dosage form structures are much slower than the dissolution rate of a drug particle that has been released from the dosage form. The disintegration rate of the dosage form structure is therefore rate-determining.

### CONCLUDING REMARKS

In conclusion, this invention discloses a micro- or milli-fluidic method and apparatus for producing cellular solids with predictable cell size and volume fraction of cells. The method and apparatus enable faster and more economical development and manufacture of high-quality and tailor-made cellular solids.

It is contemplated that a particular feature described either individually or as part of an embodiment in this disclosure can be combined with other individually described features, or parts of other embodiments, even if the other features and embodiments make no mention of the particular feature. Thus, the invention herein extends to such specific combinations not already described. Furthermore, the drawings and embodiments of the invention herein have been presented as examples, and not as limitations. Thus, it is to be understood that the invention herein is not limited to these precise embodiments. Other embodiments apparent to those of ordinary skill in the art are within the scope of what is claimed.

We claim:

1. An apparatus for the manufacture of cellular solids comprising:
An internally hollow housing having an internal surface encapsulating and defining an extrusion channel having a first end and a second end and a cross section extending axially along its length from said first end to said second end and terminating into an exit port at the second end; said housing having at least a first feeding port between the first end and second end for injecting at least one granular solid into the extrusion channel and having at least a second feeding port between the first feeding port and the exit port for injecting cell-fluid into the extrusion channel;
a granular solid feeding unit for injecting one or more granular solids through the first feeding port into the extrusion channel;
at least one heating element for fluidizing at least one injected granular solid so that the injected one or more granular solids form a plasticized matrix in the extrusion channel;
a cell-fluid injection unit attached to at least the second feeding port for injecting cell-fluid into the plasticized matrix to form a plasticized foam in the extrusion channel;
at least one conveying element for extruding the plasticized foam in the extrusion channel through an exit port; and
a mold or a surface for accepting the extruded plasticized foam.

2. The apparatus of claim 1, wherein a fraction of the housing is optically transparent.

3. The apparatus of claim 1, wherein the cell-fluid injection unit comprises a cell-fluid injection channel having a first end and at least a second end contiguous with and terminating in the extrusion channel.

4. The apparatus of claim 3, wherein the volume of the cell-fluid injection channel divided by the number of its second ends contiguous with and terminating in the extrusion channel is no greater than 200 mm³.

5. The apparatus of claim 4, wherein the cell fluid injection unit further comprises a device for the application of pressure pulses in the cell-fluid injection channel so as to control the volume of injected cells and the repetition rate of cell injection.

6. The apparatus of claim 5, wherein the device for the application of pressure pulses in the cell-fluid injection channel comprises at least one cell-fluid pump.

7. The apparatus of claim 5, wherein the device for the application of pressure pulses in the cell-fluid injection channel comprises at least an on/off valve, said on/off valve having at least an on-state and at least an off-state, said on-state permitting cell fluid injection from a pressure reservoir into the extrusion channel, said off-state restricting cell fluid injection from said pressure reservoir into the extrusion channel.

8. The apparatus of claim 5, wherein the pressure pulse duration in one repetition period is no greater than 10 seconds and wherein the repetition rate of pressure pulses is greater than 2 per minute.

9. The apparatus of claim 1, wherein the cell-fluid injection unit comprises at least one flow resistor and wherein the greater of the flow resistance of said flow resistor or the sum of the flow resistances of all the flow resistors connected in series is greater than 1×10⁹ Pa·s/m³.

10. The apparatus of claim 1 wherein at least one conveying element is a screw.

11. The apparatus of claim 1, wherein the plasticized foam solidifies in a mold or on a surface.

12. The apparatus of claim 1, wherein the cellular solid is a pharmaceutical solid dosage form.

13. An apparatus for the manufacture of cellular solids comprising:
An internally hollow housing having an internal surface encapsulating and defining an extrusion channel having a first end and a second end and a cross section extending axially along its length from said first end to said second end and terminating into an exit port at the second end; said housing having at least a first feeding port between the first end and second end for injecting at least one granular solid into the extrusion channel, at least a second feeding port between the first feeding port and the exit port for injecting at least one liquid into the extrusion channel to form a plasticized matrix by solvating the at least one injected granular solid, and at least one third feeding unit between the second feeding port and an exit port for injecting cell-fluid into the extrusion channel;
a granular solid feeding unit for injecting at least one granular solid through the first feeding port into the extrusion channel;
a solvent feeding unit attached to the second feeding port for injecting at least one solvent;
a cell-fluid feeding unit attached to at least the third feeding port for injecting cell-fluid into the plasticized matrix to form a plasticized foam in the extrusion channel;
at least one conveying element for extruding the plasticized foam in the extrusion channel through an exit port; and
a mold or a surface for accepting the extruded plasticized foam.

14. A method of manufacturing cellular solids comprising the steps of:
injecting one or more granular solids into an extrusion channel having a cross section extending along its length inside a housing, wherein at least one granular solid melts upon heating;
heating the injected one or more granular solids to form a plasticized matrix;
conveying the plasticized matrix towards an exit port of the extrusion channel by applying mechanical work on the plasticized matrix;
injecting cell-fluid into the extrusion channel to charge the plasticized matrix with cell-fluid and form a plasticized foam as the plasticized matrix transits towards an exit port;
dispensing the plasticized foam through an exit port into a mold or on a solid surface; and
solidifying the plasticized foam in said mold or on said solid surface.
wherein
the cell-fluid injection is performed using a cell-fluid injection unit having a cell-fluid injection channel with a first end and at least a second end contiguous with and terminating in the extrusion channel; and
the cell fluid is injected into a flowing plasticized matrix by the application of pressure pulses in the cell-fluid injection channel so as to control the volume of injected cells and the repetition rate of cell injection.

15. A method of manufacturing cellular solids comprising the steps of:
injecting one or more granular solids into an extrusion channel having a cross section extending along its length inside a housing;
heating the injected one or more granular solids to form a plasticized matrix or injecting a solvent to solvate at least one injected granular solid so that the one or more injected granular solids form a plasticized matrix;
conveying the plasticized matrix towards an exit port of the extrusion channel by applying mechanical work on the plasticized matrix;
injecting cell-fluid into the extrusion channel to charge the plasticized matrix with cell-fluid and form a plasticized foam as the plasticized matrix transits towards an exit port; and
dispensing the plasticized foam through an exit port into a mold or on a surface.

16. The method of claim 15, wherein the cell-fluid injection is performed using a cell-fluid injection unit having a cell-fluid injection channel with a first end and at least a second end contiguous with and terminating in the extrusion channel.

17. The method of claim 16, wherein the volume of the cell-fluid injection channel divided by the number of its second ends contiguous with and terminating in the extrusion channel is no greater than 200 mm³.

18. The method of claim 16, wherein the cell fluid is injected into a flowing plasticized matrix by the application of pressure pulses in the cell-fluid injection channel so as to control the volume of injected cells and the repetition rate of cell injection.

19. The method of claim 18, wherein the device for the application of pressure pulses in the cell-fluid injection channel comprises at least one cell-fluid pump.

20. The method of claim 18, wherein the device for the application of pressure pulses in the cell-fluid injection channel comprises at least an on/off valve, said on/off valve having at least an on-state and at least an off-state, said on-state permitting cell fluid injection from a pressure reservoir into the extrusion channel, said off-state restricting cell fluid injection from said pressure reservoir into the extrusion channel.

21. The method of claim 18, wherein the pressure pulse duration in one repetition period is no greater than 10 seconds and wherein the repetition rate of pressure pulses is greater than 2 per minute.

22. The method of claim 15, wherein the cell-fluid injection is performed using at least one flow resistor and wherein the greater of the flow resistance of said flow resistor or the sum of the flow resistances of all the flow resistors connected in series is greater than 1×10⁹ Pa·s/m³.

23. The method of claim 15 wherein the mechanical work is applied using a screw.

24. The method of claim 15, wherein the plasticized foam solidifies in a mold or on a surface.

25. The method of claim 15, wherein the cellular solid is a pharmaceutical solid dosage form.
